(19) United States
(12) Patent Application Publication
(10) Pub. No.: US 2024/0327923 A1
(43) Pub. Date: Oct. 3, 2024

(54) Marker Set and Its Use for the Identification of a Disease Based on PCL-Like Transcriptomic Status

(71) Applicant: Erasmus University Medical Center Rotterdam, Rotterdam (NL)

(72) Inventors: Davine Hofste Op Bruinink, Stockholm (SE); Rowan Kuiper, Rotterdam (NL); Pieter Sonneveld, Schiedam (NL)

(21) Appl. No.: 18/293,409

(22) PCT Filed: Aug. 5, 2022
(86) PCT No.: PCT/NL2022/050460

§ 371 Date: Jan. 30, 2024

(30) Foreign Application Priority Data
Aug. 6, 2021 (EP) 21190230.9

(51) Int. Cl. C12Q 1/6886

(52) U.S. Cl. CPC C12Q 1/6886; C12Q 2600/112; C12Q 2600/118; C12Q 2600/158

(57) ABSTRACT

The present invention refers to a marker set for determining a PCL-like transcriptomic status in a sample which is indicative for a disease. Further, a method for determining a PCL-like transcriptomic status in a sample is provided by the present invention. In addition, the marker set and/or the method of the present invention is used for selecting an active agent for use in the treatment and/or prevention of a disease. In addition, the present invention refers to kits comprising means for determining the PCL-like transcriptomic status based on the marker set in a sample.

The present invention refers to a marker set and its use in identifying a disease based on the determination a PCL-like transcriptomic status in a sample. The marker set of the present invention is for example also used for selecting an active agent for use in the treatment of a disease. Further, the present invention is directed to kits comprising means for determining the PCL-like transcriptomic status in a sample as well as selecting an active agent based thereon.

### TECHNICAL BACKGROUND

Plasma cells, also called plasma B cells, are a type of white blood cells that originate in the lymphoid organs by B lymphocytes and secrete antibodies. Plasma cells may develop plasma cell dyscrasias which constitute various plasma cell disorders ranging from benign to malignant conditions, eventually resulting in the degeneration of plasma cells.

Among plasma cell dyscrasias, multiple myeloma (MM), also known as plasma cell myeloma and simply myeloma, represents a cancer of plasma cells. The cause of MM is unknown. Risk factors include for example obesity, radiation exposure, family history, and certain chemicals. MM is considered generally incurable, however, treatable.

Metastatic capacity is a pivotal feature of aggressive cancers, for which tumor cell dissemination is an early requirement. Since the PCL-like classifier can identify plasma cell tumors that have a higher degree of hematogenous dissemination than would be expected based on tumor burden alone and many pathways are represented in this classifier that are part of known cancer hallmarks (Hanahan & Weinberg—Cancer Cell 2011), it may be anticipated that the PCL-like classifier will have prognostic value in other malignancies and pre-malignant conditions as well.

Plasma cell leukemia (PCL) is the most aggressive form of plasma cell dyscrasias and thus, represents a very serious and therapeutically challenging disease. Around 2% of all plasma cell dyscrasias are PCL.

PCL may present as primary plasma cell leukemia (pPCL), i.e. in patients without prior history of a plasma cell dyscrasia or as secondary plasma cell leukemia (sPCL), i.e. in patients previously diagnosed with a history of its predecessor dyscrasia such as MM.

For over a century, the level of circulating tumor cells (CTCs) has been assessed in MM to identify PCL. Even though MM is characterized by an intramedullary outgrowth of malignant plasma cells, the degree of hematogenous tumor cell dissemination is highly variable between patients. At the time of diagnosis, CTCs are routinely quantified in peripheral blood (PB) by morphology and can be detected in the majority of MM patients if flow cytometry is used. However, in only 2% of patients these levels are ≥20% or ≥2×10⁹/L, which is pathognomonic for pPCL.

Symptomatic MM patients with lower CTC levels at diagnosis are classified as newly diagnosed MM (NDMM), but these may still develop sPCL after treatment.

Clinically, pPCL is considered a high-risk disease entity within MM. pPCL patients commonly present with a large tumor burden and extensive morbidity, show poor response to standard treatment and have a dismal overall survival.

Disease aggressiveness in pPCL is considered to be reflected by the presence of significantly higher CTC levels than in NDMM. Even though this was previously hypothesized to be the result of a spill over from a large intramedullary tumor, evidence is accumulating that altered molecular features involved in cell adhesion, evasion of apoptosis, migration, bone marrow (BM) independence and RNA metabolism are associated with this phenotype.

Yet, several reports have suggested that certain NDMM patients experience an equally aggressive disease course to that of pPCL, without having CTC levels ≥20%. Such NDMM patients are diagnosed as PCL-like MM.

Still, molecular determinants remain poorly understood, with conventional prognostic risk markers in NDMM (i.e. t (4;14), t (14;16) and deletion of chromosome 17p (del17p)) only being detectable in a subset of pPCL tumors.

Thus, a problem to be solved is for example the provision of means and methods to reliably and specifically identify a disease, for example a rare disease and/or a high grade of a disease, in a sample.

The present invention provides for the first time a marker set based on analysis of the transcriptomic profile for molecularly identifying diseases, for example cancer diseases, such as pPCL.

The present invention provides a marker set which has independent prognostic value in the context of conventional risk markers. The present invention facilitates for example a high sensitivity (93%) to detect pPCL, but also identified PCL-like MM in 11% of NDMM patients.

Hence, the present invention provides a marker set and methods for the determination of a novel and efficient high-risk biology that is, for example, already detectable in NDMM patients, despite not being clinically leukemic. Moreover, the present invention significantly improves the accuracy in diagnostics and treatment of rare diseases as well as the prognostic performance in the context of such disease.

### SUMMARY OF THE INVENTION

The present invention refers to a marker set for determining a PCL-like transcriptomic status in a sample which is indicative for a disease, wherein the marker set comprises coding or non-coding genes associated to biological pathways and/or chromosomal location. The marker set according to the present invention indicates for example a rare disease and/or a high grading of the disease.

The marker set of the present invention is for example selected from the group consisting of cell adhesion marker, immune response marker, cell metabolism marker, tumor suppression marker, post-translational protein modification marker, (post-) transcriptional regulation marker, cellular (matrix) structure marker, cell migration marker, cell death marker, cell signaling marker, protein biogenesis and transport marker, cell proliferation marker, DNA damage response marker, or a combination thereof (see e.g., Hofste op Bruinink et al., J Clin Oncol 2022; Chakraborty & Lentzsch, J Clin Oncol 2022).

The marker set is for example selected from two or more or optionally all from the group of markers consisting of SDC1, IGLV3-19, PPAPDC1B, WDR11, ALG14, PHF19, TSC22D1, FAM174A, TSPAN3, CALU, TPM1, VCAM1, IDH2, P2RY6, ASAH1, IGHV1-69, FUCA1, STRN, CYSTM1, APH1B, SLAMF7, YIPF5, APOE, SPATS2, PRKCA, PSME4, SLFN11, RMDN3, CHID1, TMEM45A, TARSL2, DCLRE1C, TCTN3, DAP, DCK, SMOC1, EMC7, LINC00582, KDELR1, APOBEC3B, CRTAP, BRSK1, MZB1, ERI3, DERL3, CENPM, GDE1, FLNA, NCF4, DNASE1L3, ITGA8, SELENOM, AL159169.2, AC092620.1, or a combination thereof.

A sample according to the present invention is for example selected from plasma cell, blood, (pre-) malignant plasma cell, bone marrow, urine, serum, cells and tissue such as tumor tissue or tumor cells, or a combination thereof. In some embodiments the sample is from an individual afflicted with multiple myeloma.

The present invention also refers to a method for determining a PCL-like transcriptomic status in a sample which is indicative for a disease comprising the steps of
a) isolating RNA from the sample
b) determining the expression profile of the marker set according to the present invention in the isolated RNA,
c) calculating a score, wherein the score is based on the first principal component of the expression profile of the marker set in a classifier's discovery data,
d) comparing the score calculated in step c) to a reference score.

The score calculated in step c) is for example the lowest score that at least 90 to 100% of the samples in a reference have a higher score. For example, the score of step c) in the range of at least 1 to 7 is indicative for a disease corresponding to the disease of the reference of step d).

The method of the present invention further comprises for example the steps of
e) determining the CTC level in the sample, and
f) optionally determining the tumor burden
g) referencing the expression profile of step b) to the CTC level or to the CTC level referenced to the tumor burden.

The tumor burden is for example determined based on the percentage of plasma cells in bone marrow, M-protein in serum and/or urine, the level of beta-2 microglobulin in serum, the level of lactate dehydrogenase in serum, by imaging, or a combination thereof.

The method optionally further comprises classifying the sample as having a high or standard SKY92 risk status, comprising determining in the sample the expression profile of each marker listed in Table 7.

The present invention further refers to a method for determining a treatment or prognosis for an individual afflicted with multiple myeloma, comprising:
determining a PCL-like transcriptomic status in a sample from said individual according to a method of any one of claims 7-12,
determining the SKY92 risk status in a sample from said individual, comprising determining in the sample the expression profile of each marker listed in Table 7, and classifying the individual as having a high or standard SKY92 risk status.

In addition, the present invention is directed to a method for treating an individual afflicted with multiple myeloma, comprising:
determining a PCL-like transcriptomic status in a sample from said individual according to the methods of the present invention, and
treating the individual by providing a cancer treatment to said individual.

Moreover, the present invention relates to a method for treating an individual afflicted with multiple myeloma, comprising:
a) determining a PCL-like transcriptomic status in a sample from said individual according to the methods of the present invention,
b) determining the SKY92 risk status in a sample from said individual, comprising determining in the sample the expression profile of each marker listed in Table 7,
c) classifying said individual as having a PCL-like transcriptomic status and/or having a SKY92 high risk status, and
d) treating the individual of step c) by providing a cancer treatment to said individual.

In the methods for treating an individual afflicted with multiple myeloma of the present invention an individual is for example classified as having a PCL-like transcriptomic status and optionally a SKY92 high risk status is intensively monitored, and the individual is treated with quadruplet induction therapy including anti-CD38, high dose autologous stem cell transplantation therapy or a combination thereof. In these methods for example a bispecific antibody, a CAR T cell or a combination thereof is administered.

The PCL-like transcriptomic status determined by the method of the present invention indicates for example a high grading of a disease which correlates to at least one prognostic risk model. The at least one prognostic risk model is specific for the disease. For example the prognostic risk model is selected from the group consisting of R-ISS status, ISS status, FISH status, SKY92 status, UAMS70 status of NDMM, or a combination thereof.

The method of the present invention further comprises for example selecting an active agent, such as a chemotherapeutic, for treatment of a disease based on the PCL-like transcriptomic status in a sample.

The marker set or the method of the present invention indicates for example a disease selected from the group consisting of newly diagnosed multiple myeloma (NDMM), primary plasma cell leukemia (pPCL), secondary plasma cell leukemia (pPCL), progressive disease (PD), smoldering multiple myeloma (SMM), monoclonal gammopathy of undetermined significance (MGUS), plasmacytomas, Waldenström's macroglobulinemia, POEMS syndrome, breast cancer, lung cancer, malignant melanoma, lymphoma, skin cancer, bone cancer, prostate cancer, liver cancer, brain cancer, cancer of the larynx, gall bladder, pancreas, testicular, rectum, parathyroid, thyroid, adrenal, neural tissue, head and neck, colon, stomach, bronchi, kidneys, basal cell carcinoma, squamous cell carcinoma, metastatic skin carcinoma, osteo sarcoma, Ewing's sarcoma, reticulum cell sarcoma, liposarcoma, myeloma, giant cell tumor, small-cell lung tumor, islet cell tumor, primary brain tumor, meningioma, acute and chronic lymphocytic and granulocytic tumors, acute and chronic myeloid leukemia, hairy-cell tumor, adenoma, hyperplasia, medullary carcinoma, intestinal ganglioneuromas, Wilms tumor, seminoma, ovarian tumor, leiomyomatous tumor, cervical dysplasia, retinoblastoma, soft tissue sarcoma, malignant carcinoid, actinic keratosis, melanoma, pancreatic cancer, colon cancer, rhabdomyosarcoma, Kaposi's sarcoma, osteogenic sarcoma, malignant hypercalcemia, renal cell tumor, polycythermia vera, myeloproliferative disease, essential thrombocytosis, lymphoma, mastocytosis, myelodysplastic syndrome, clonal hematopoiesis of indeterminate potential, monoclonal B-cell lymphocytosis, chronic myelomonocytic leukemia, myelofibrosis, adenocarcinoma, anaplastic astrocytoma, glioblastoma multiforma, epidermoid carcinoma, a disease characterized by a circulating tumor cell, such as a circulating malignant plasma cell, or a combination thereof.

Furthermore, the present invention relates to a kit for determining a PCL-like transcriptomic status which is indicative for a disease comprising, probes, primers, or a combination thereof for determining an expression profile of a marker set of the present invention in a sample, optionally means for determining the CTC level in a sample and optionally means for determining the tumor burden in a sample. Optionally, the kit further comprises an active agent for use in a method of treating the disease detected by the marker. The expression profile of a marker set according to the present invention is for example determined using a microarray, next generation sequencing or qRT-PCR.

### DESCRIPTION OF FIGURES

FIG. 1 shows a CONSORT diagram illustrating an overview of patients useful to include in the marker set screen of the present invention. CTC level, tumor transcriptomic and tumor burden data from cohort 1 were used to construct and validate the marker set of the present invention. Transcriptomic profiling and follow-up data from cohort 2 were leveraged to determine the prevalence of PCL-like MM in a wide range of plasma cell samples (prevalence cohort), as well to test its prognostic value in NDMM (survival cohort).

FIGS. 2A and 2B show an overview of baseline CTC levels and timing of flow cytometric CTC quantification. (2A) Histogram showing baseline CTC levels and timing of CTC quantification of n=297 NDMM patients. In 282/297 (95%) patients, CTC levels were determined on the same or next day after sampling. (2B) Scatterplot of all 40/297 (13%) CTC samples from NDMM patients that had a CTC level under the detection limit. Dots represent the number of leukocytes that have been measured per CTC sample and the corresponding limit of detection. The dashed line indicates a limit of detection of 1×10⁻⁵, i.e. 1 CTC in 100,000 leukocytes. The color of the dots reflects the timing of the CTC quantification after sampling.

FIG. 3A to 3E show clinical and molecular determinants of pPCL. (3A) Boxplot showing CTC levels in pPCL (n=51) and NDMM patients with detectable CTC levels (n=257) from cohort 1, using a two-sided Wilcoxon test for comparison. Data are shown on a log (odds) scale. The bold bars in the boxplots correspond to the median CTC level per disease stage, the lower and upper hinges to the first and third quartiles. The whiskers extend to 1.5 times the interquartile range at most; data points beyond this level are depicted as outliers, represented by black dots. (3B) Boxplot showing baseline tumor burden data between pPCL (n=50) and NDMM patients (n=271) from cohort 1, using a two-sided Wilcoxon test for comparison. (3C) Combined scatter and density plot of tumor burden and CTC level data in NDMM patients with detectable CTC levels (n=235) and pPCL (n=50) from cohort 1. The dashed line represents the fitted linear model of the association between CTC level and tumor burden data, with the corresponding adjusted correlation coefficient and p-value indicated in the left upper corner. Data are shown on a log (odds) scale. (3D) Clinical, cytogenetic and immunophenotypic baseline characteristics of pPCL (n=51) and NDMM patients (n=297) from cohort 1. Associations of baseline characteristics with disease stage were determined with a Fisher's exact test, whereas the association with CTC level and tumor burden was tested by fitting a linear model. All p-values were corrected for multiple testing according to the Benjamini-Hochberg procedure. (3E) Global principal component analysis plot of all available transcriptomic profiles of pPCL (n=29) and NDMM (n=154) BM tumor samples from cohort 1, using all n=12,928 expressed genes as input.

FIG. 4A to 4D show the construction and validation of a marker set according to the present invention. (4A) Volcano plot showing all n=12,928 expressed genes of which the association with a high CTC level was tested in the discovery cohort (n=95 NDMM and n=15 pPCL patients), by applying a linear regression model including tumor burden as additional covariate, followed by correction for multiple testing according to the Benjamini-Hochberg procedure. The log fold change corresponds to the change in gene expression per log (odds) unit increase in CTC level, independent of tumor burden. N=1700 genes showed a significant association and are depicted in color (FDR<0.05). The open circles represent the n=54 most significant genes that have been selected for the PCL-like classifier. Their corresponding normalized expression values are shown in the heatmap for all available pPCL (n=29) and NDMM (n=154) BM tumor transcriptomes in cohort 1. Gene names are displayed according to the HUGO Gene Nomenclature that corresponds with Ensembl release 74. Gene names that were matched based on a later release of Ensembl are indicated with an asterisk. (4B) Scatter plot showing the association between the score and CTC level in the discovery cohort (n=116 patients), as determined with a linear regression model. The dashed line represents the lowest score of pPCL samples in the discovery cohort (3.55), which is the threshold for the PCL-like classifier. NDMM samples with a score ≥3.55 are classified as PCL-like MM; NDMM samples with a score <3.55 are classified as i-MM. CTC level is displayed on a log (odds) scale. (4C) Scatter plot showing the association between PCL-like score and CTC level in the validation cohort (n=57 patients), as determined with a linear regression model. The dashed line represents the threshold of the PCL-like classifier above which samples are classified as PCL-like. CTC level is displayed on a log (odds) scale. (4D) Combined scatter and density plots of tumor burden, CTC level and disease subtype data for all patients from cohort 1 with available data (n=121 i-MM, n=13 PCL-like MM, n=28 pPCL patients). The adjusted correlation coefficient and p-value represent the association between BM plasmacytosis and CTC level, as determined with a linear regression model. In the density plots, PCL-like MM was compared with i-MM and pPCL, respectively, using a two-sided Wilcoxon test. Only significant differences are shown.

FIG. 5A to 5C show gene selection for the PCL-like classifier.

(5A) Line chart displaying the significance of the difference in scores between NDMM (n=109) and pPCL samples (n=15) in the discovery cohort, as determined with a two-sided Wilcoxon test for each number of genes in the classifier ranging from 25 to 422. Genes were previously selected and ranked based on the significance of their association with CTC levels. The dashed line represents the number of genes with which the highest significance was reached between scores of NDMM versus pPCL samples. (5B) Line chart representing the score per sample in the discovery cohort, computed over a range of gene numbers in the classifier. Per sample and per number of genes in the classifier, a score was computed according to a leave-one-out cross-validation procedure, as described in detail in the Examples. (5C) Principal component analysis plot using the centered expression values of 54 genes identified in the previous steps as input. PC1 represents the score that was determined on all n=124 samples from the discovery cohort and projected on all n=59 samples from the validation cohort.

FIG. 6A to 6C show the concordance of risk classification on paired microarray versus RNA Seq data. Scatter plots of paired transcriptomic profiles generated on both microarray and RNA Seq platforms from n=123 NDMM BM tumor samples. Data were processed as outlined in detail in the Supplementary Methods, after which scores according to the method of the present invention (6A), SKY92 scores (6B) and UAMS70 scores (6C) were computed for all samples. Adjusted correlation coefficients and p-values represent the association of paired risk scores, as assessed with a linear regression model. Colored quadrants within the scatter plots represent the proportion of samples classified as high-risk with either platform.

FIGS. 7A and 7B show CTC level prediction based on the score with tumor burden. (7A) Scatterplot of observed versus predicted CTC levels for all patients with detectable CTC levels and available tumor burden data in the discovery cohort (n=110). The dashed line represents the corresponding regression line. Predicted CTC levels were estimated based on a formula that was derived from fitting score and tumor burden data to a linear regression model with observed CTC levels. The corresponding adjusted correlation coefficient and p-value are displayed in the upper left corner of the plot. (7B) Scatterplot of observed versus predicted CTC levels for all patients with detectable CTC levels and available tumor burden data in the validation cohort (n=52).

FIG. 8A to 8E show clinical and molecular determinants of PCL-like MM. (8A) Violin plot of PCL-like scores from healthy plasma cell, MGUS, SMM, NDMM and pPCL BM tumor samples from the prevalence cohort (n=1801 patients), comprising 10 different datasets. (8B) Density plot showing the number of differentially expressed ssGSEA pathways (FDR<0.05) per comparison between PCL-like versus pPCL and i-MM versus pPCL samples from the prevalence cohort (n=757 i-MM, n=99 PCL-like MM, n=29 pPCL samples). With a linear model, ssGSEA scores of n=1788 pathways were compared between n=29 pPCL and a random sample of n=29 i-MM or PCL-like samples, which was performed n=1000 times. (8C) Box plots of ten pathways that were most significantly upregulated in PCL-like MM (n=99) versus i-MM samples (n=757) from the prevalence cohort with a logFC >0.75 (FDR<0.05), displayed per disease subtype. The bold bars in the boxplots correspond to the median normalized ssGSEA scores per disease subgroup, the lower and upper hinges to the first and third quartiles. The whiskers extend to 1.5 times the interquartile range at most; data points beyond this level are depicted as outliers, as represented by black dots. (8D) Box plots of ten pathways that were most significantly downregulated in PCL-like MM (n=99) versus i-MM samples (n=757) from the prevalence cohort, with a logFC<−0.75 (FDR<0.05), displayed per disease subtype. (8E) Histograms comparing baseline characteristics of PCL-like MM with pPCL, as well as of i-MM with pPCL (prevalence cohort). The Fisher's exact test was used for comparisons, followed by correction for multiple testing according to the Benjamini-Hochberg procedure. Error bars represent the 95% confidence interval of the observed prevalence per disease subgroup, as determined with the Wilcoxon score interval with continuity correction.

FIG. 9A to 9C show prevalence of PCL-like transcriptomic status at the time of progression, in extramedullary disease and in different transcriptional clusters. (9A) Violin plots of the prevalence of PCL-like disease, in CTCs and in cell lines, with the number of samples per PCL-like transcriptomic status shown for each relevant patient cohort from the prevalence cohort. (9B) Violin plots of the prevalence of PCL-like disease per transcriptional cluster in NDMM (n=1694 patients, prevalence cohort). (9C) Violin plots of the prevalence of PCL-like disease per transcriptional cluster in pPCL (n=29 patients, prevalence cohort).

FIGS. 10A and 10B show meta-analysis of univariate prognostic significance of the PCL-like classifier in NDMM. (10A) Kaplan-Meier plots, risk table and forest plot of the association of PCL-like transcriptomic status with progression-free survival in NDMM in seven different patient cohorts from six trials (n=1540 patients). The difference in survival between PCL-like MM and i-MM was computed with the logrank test. Dashed lines in the Kaplan-Meier plots represent the median survival of PCL-like MM patients per trial cohort, with the median survival shown on the right. The output of the meta-analysis according to a random effects model was used as input for the forest plot. The size of the boxes represents the relative size of the patient cohorts; the whiskers represent the 95% confidence interval of the estimated hazard ratio for progression of PCL-like MM versus i-MM. The dashed line in the forest plot represents the overall hazard ratio. The diamond represents the overall estimated hazard ratio with the 95% confidence interval. (10B) Kaplan-Meier plots, risk table and forest plot of the association of PCL-like transcriptomic status with overall survival in NDMM in seven different patient cohorts from six trials (n=1540 patients).

FIG. 11A to 11E show multivariate analysis of the association of PCL-like transcriptomic status with overall survival in NDMM. Kaplan-Meier plots of the association of PCL-like transcriptomic status with overall survival in combination with conventional prognostic risk models in NDMM from the survival cohort. P-values represent the prognostic significance of the overall model, as determined with the logrank test. (11A) PCL-like transcriptomic status in combination with R-ISS status. (11B) PCL-like transcriptomic status in combination with ISS status. (11C) PCL-like transcriptomic status in combination with high-risk FISH status. (11D) PCL-like transcriptomic status in combination with SKY92 high-risk status. (11E) PCL-like transcriptomic status in combination with UAMS70 high-risk status.

FIG. 12A to 12E show multivariate analysis of the association of PCL-like transcriptomic status with progression-free survival in NDMM. Kaplan-Meier plots of the association of PCL-like status with progression-free survival in combination with conventional prognostic risk models in NDMM. P-values represent the prognostic significance of the overall model, as determined with the logrank test. (12A) PCL-like transcriptomic status in combination with R-ISS status. (12B) PCL-like status in combination with ISS status. (12C) PCL-like transcriptomic status in combination with high-risk FISH status. (12D) PCL-like transcriptomic status in combination with SKY92 high-risk status. (12E) PCL-like transcriptomic status in combination with UAMS70 high-risk status.

FIG. 13 shows positive predictive value and sensitivity to detect PCL-like transcriptomics in NDMM, in the context of clinically relevant CTC level thresholds. Schematic overview of the association between PCL-like transcriptomics of BM tumor cells, CTC levels and tumor burden in our study cohort. Horizontal dashed line represents the clinically relevant CTC level threshold of 20% in MM. Patients were consistently classified based on CTC levels that were equal or higher than the given threshold versus lower. The vertical dashed line represents the PCL-like score threshold that is used to distinguish a PCL-like transcriptome from an intramedullary transcriptome. The honeycombs represent MM patients and show the positive association of CTC level with both PCL-like score and tumor burden that was identified in this study. Moreover, pPCL patients were found to have a similar BM tumor transcriptome to PCL-like MM patients, but with generally a higher tumor burden. In our cohort, 80% of NDMM patients with >2-5% CTCs had a PCL-like transcriptome of their BM tumor cells. However, with these CTC level thresholds a large proportion (47-73%) of all NDMM patients with a PCL-like tumor transcriptome would be missed. On the contrary, prognostically relevant CTC level thresholds in NDMM (>0.02-0.27%) showed a high sensitivity (87-100%) to identify NDMM patients with a PCL-like tumor transcriptome, but this also corresponded with a low positive predictive value (16-34%) for a PCL-like tumor transcriptome among NDMM patients with CTC levels at or above these thresholds.

### DETAILED DESCRIPTION

The present invention provides a marker set for determining a plasma cell leukemia like (PCL-like) transcriptomic status in a sample which is indicative for a disease. The present invention is further directed to a method as well as kits for determining a PCL-like transcriptomic status in a sample which is indicative for a disease.

In addition, the present invention forms the basis for use of the marker set in identifying a disease for selecting an active agent, e.g., a chemotherapeutic or an antagonist or agonist modulating, i.e., decreasing or increasing, the expression of one or more genes of the marker set of the present invention, and therapy for preventing and/or treating the disease, respectively.

In the following, the features of the present invention will be described in more detail. It should be understood that embodiments may be combined in any manner and in any number to create additional embodiments. The variously described examples and embodiments should not be construed to limit the present invention to only the explicitly described embodiments. This description should be understood to support and encompass embodiments which combine the explicitly described embodiments with any number of the disclosed features. Furthermore, any permutations and combinations of all described features in this application should be considered disclosed by the description of the present application unless the context indicates otherwise.

Throughout this specification and the claims, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps. The terms “a” and “an” and “the” and similar reference used in the context of describing the invention (especially in the context of the claims) are to be construed to cover both the singular and the plural, unless otherwise indicated herein or clearly contradicted by the context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”, “for example”), provided herein is intended merely to better illustrate the invention and does not pose a limitation on the scope of the invention otherwise claimed. No language in the specification should be construed as indicating any non-claimed element essential to the practice of the invention.

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

The marker set of the present invention determines a PCL-like transcriptomic status in a sample which is indicative for a disease. Any disease which is associated to the PCL-like transcriptomic status is identified by the marker set and method of the present invention. Accordingly, such diseases are equally designated as PCL-like diseases.

The PCL-like transcriptomic status determined by the marker set or method of the present invention is indicative for rare diseases or a high grading of a disease. A high grading of a disease is considered as a high-risk disease causing high morbidity, low overall survival (OS) and low progression free survival (PFS). A high grading of a disease also comprises high-risk cancers which are further characterized to recur (come back), or spread. A rare disease also comprises severe diseases and/or a high grade of a disease. In an exemplary embodiment, the presence of a PCL-like transcriptomic status in a sample from an individual afflicted with multiple myeloma classifies said individual as having a poor prognosis.

The marker set and the method of the present invention determines a PCL-like transcriptomic status in a sample which is indicative for several diseases. Such diseases for example are selected from the group consisting of newly diagnosed multiple myeloma (NDMM), primary plasma cell leukemia (pPCL), secondary plasma cell leukemia (pPCL), progressive disease (PD), smoldering multiple myeloma (SMM), monoclonal gammopathy of undetermined significance (MGUS), plasmacytomas, Waldenström's macroglobulinemia, POEMS syndrome, breast cancer, lung cancer, malignant melanoma, lymphoma, skin cancer, bone cancer, prostate cancer, liver cancer, brain cancer, cancer of the larynx, gall bladder, pancreas, testicular, rectum, parathyroid, thyroid, adrenal, neural tissue, head and neck, colon, stomach, bronchi, kidneys, basal cell carcinoma, squamous cell carcinoma, metastatic skin carcinoma, osteo sarcoma, Ewing's sarcoma, reticulum cell sarcoma, liposarcoma, myeloma, giant cell tumor, small-cell lung tumor, islet cell tumor, primary brain tumor, meningioma, acute and chronic lymphocytic and granulocytic tumors, acute and chronic myeloid leukemia, hairy-cell tumor, adenoma, hyperplasia, medullary carcinoma, intestinal ganglioneuromas, Wilms tumor, seminoma, ovarian tumor, leiomyomatous tumor, cervical dysplasia, retinoblastoma, soft tissue sarcoma, malignant carcinoid, actinic keratosis, melanoma, pancreatic cancer, colon cancer, rhabdomyosarcoma, Kaposi's sarcoma, osteogenic sarcoma, renal cell tumor, polycythemia vera, myeloproliferative disease, essential thrombocytosis, lymphoma, mastocytosis, myelodysplastic syndrome, clonal hematopoiesis of indeterminate potential, monoclonal B-cell lymphocytosis, chronic myelomonocytic leukemia, myelofibrosis, adenocarcinoma, anaplastic astrocytoma, glioblastoma multiforma, epidermoid carcinoma, a disease characterized by a circulating tumor cell, such as a circulating malignant plasma cell, or a combination thereof.

The marker set of the present invention is for example selected from coding or non-coding genes. Such genes are for example associated to biological pathways and/or a chromosomal location.

For example the marker set is selected from the group consisting of adhesion marker, immune response marker, cell metabolism marker, tumor suppression marker, post-translational protein modification marker, (post-) transcriptional regulation marker, cellular (matrix) structure marker, cell migration marker, cell death marker, cell signaling marker, protein biogenesis and transport marker, cell proliferation marker, DNA damage response marker, or a combination thereof.

For example, the marker set is selected from the group of markers as shown in Table 5 consisting of SDC1, IGLV3-19, PPAPDC1B, WDR11, ALG14, PHF19, TSC22D1, FAM174A, TSPAN3, CALU, TPM1, VCAM1, IDH2, P2RY6, ASAH1, IGHV1-69, FUCA1, STRN, CYSTM1, APH1B, SLAMF7, YIPF5, APOE, SPATS2, PRKCA, PSME4, SLFN11, RMDN3, CHID1, TMEM45A, TARSL2, DCLRE1C, TCTN3, DAP, DCK, SMOC1, EMC7, LINC00582, KDELR1, APOBEC3B, CRTAP, BRSK1, MZB1, ERI3, DERL3, CENPM, GDE1, FLNA, NCF4, DNASE1L3, ITGA8, SELENOM, AL159169.2, AC092620.1, or combinations thereof.

The marker set of the present invention comprises for example a combination of two or more markers selected from the groups as disclosed above. The marker set for example comprises a combination of three or more, four or more, five or more, six or more, seven or more, eight or more, nine or more, 10 or more, 13 or more, 15 or more, 20 or more, 25 or more, 30 or more, 35 or more, 40 or more, 45 or more, 50 or more, 55 or more, 60 or more, 65 or more, 70 or more, 75 or more, 80 or more, 85 or more, 90 or more, 95 or more, 100 or more, 125 or more, 150 or more, 175 or more, 200 or more, 200 or more markers selected from the groups as disclosed above. It is clear to the skilled person that selecting two or more comprises selecting all markers.

For example, the marker set comprises all markers selected from the group consisting of SDC1, IGLV3-19, PPAPDC1B, WDR11, ALG14, PHF19, TSC22D1, FAM174A, TSPAN3, CALU, TPM1, VCAM1, IDH2, P2RY6, ASAH1, IGHV1-69, FUCA1, STRN, CYSTM1, APH1B, SLAMF7, YIPF5, APOE, SPATS2, PRKCA, PSME4, SLFN11, RMDN3, CHID1, TMEM45A, TARSL2, DCLREIC, TCTN3, DAP, DCK, SMOC1, EMC7, LINC00582, KDELR1, APOBEC3B, CRTAP, BRSK1, MZB1, ERI3, DERL3, CENPM, GDE1, FLNA, NCF4, DNASE1L3, ITGA8, SELENOM, AL159169.2, AC092620.1.

The PCL-like transcriptomic status in a sample refers to an expression profile determined by the marker set of the present invention. The expression profile of the marker set according to the present invention is determined in a sample by measuring the individual expression levels of each marker comprised in the marker set of the present invention. It is clear to the skilled person that a marker set comprises single markers which for example represent genes.

An expression level for example refers to detectable nucleic acid molecules. The nucleic acid molecules are for example detected by probes, primers or combinations thereof. Development and identification of such probes and/or primers facilitating specific binding and detection of the nucleic acid molecules of the marker set according to the present invention is performed according to the standard methods known to a person skilled in the art.

The expression level of nucleic acid molecules are determined by any method known in the art including for example RT-PCT, quantitative PCR, Northern blotting, gene sequencing, in particular RNA sequencing, for example Next Generation Sequencing (NGS), and gene expression profiling techniques, such as multiplex chip techniques such as microarray.

For example the nucleic acid molecule is RNA, such as mRNA and/or pre-mRNA or DNA, such as cDNA. The level of RNA or DNA expression determined is detected directly or indirectly, for example by generating cDNA and/or by amplifying the RNA/cDNA.

General methods for RNA extraction are well known in the art and are disclosed in standard textbooks of molecular biology, including Ausubel et al. (1997) Current Protocols of Molecular Biology, John Wiley and Sons. Methods for RNA extraction from paraffin embedded tissues are disclosed, for example, in Rupp & Locker (1987) Lab Invest. 56: A67, and De Andres et al., BioTechniques 18:42044 (1995). For example, RNA isolation can be performed using purification kit, buffer set and protease from commercial manufacturers, such as Qiagen, according to the manufacturer's instructions (QIAGEN Inc., Valencia, Calif.). For example, total RNA from cells in culture can be isolated using Qiagen RNeasy mini-columns. Numerous RNA isolation kits are commercially available and can be used in the methods of the invention.

The expression levels of the marker set for example refers to the protein levels translated from the mRNAs of the markers comprised in the marker set of the present invention. Determining the expression levels of the marker set by protein detection may be performed by any method known in the art including ELISA, immunocytochemistry, flow cytometry, Western blotting, proteomic as well as mass spectrometry. Protein detection as used herein may include detection of full-length proteins, truncated proteins, peptides, polypeptides and combinations thereof.

General methods for protein purification are well known in the art and are disclosed in standard textbooks of molecular biology, including Ausubel et al. (1997) Current Protocols of Molecular Biology, John Wiley and Sons. For example, protein purification can be performed using purification kit, buffer set and protease from commercial manufacturers.

The expression level is an absolute value, but a “normalized” expression level. Normalization refers adjusting levels measured on different scales to a notionally common scale, optionally prior to averaging. Normalization is particularly useful when expression is determined based on microarray data. Normalization facilitates the correction of variations for example within microarrays and across samples so that data from different chips can be simultaneously analyzed. The robust multi-array analysis (RMA) algorithm is optionally used to pre-process probe set data into gene expression levels for all samples. (Irizarry R A, et al., Biostatistics (2003) and Irizarry R A, et al., Nucleic Acids Res. (2003)). In addition, Affymetrix's default preprocessing algorithm (MAS 5.0), is optionally also employed. Additional methods of normalizing expression data are described in US20060136145.

For example, the levels of expression can be normalized against housekeeping or another reference gene expression. For example, in microarray data, specific normalization methods for background correction, probe summarization into exon, transcript or gene level expression values and scaling of the within and/or between array expression values are employed depending on the array platform manufacturer. For example, in Affymetrix microarray data, the MAS5 algorithm is employed. Optionally, the MAS5 scaling step is replaced by other methods such as loess transformation, quantile normalization, variance stabilizing normalization, (robust) spline normalization, or others.

For example, in RNAseq, standard mapping and/or quantification software like Salmon, Kallisto, or others are employed to obtain values reflecting the log scaled expression levels of the marker set (e.g. in terms of TPM, RPKM, FPKM, counts, etc.).

For applicability to the classifier, these normalized values are optionally additionally normalized in order to be compatible with the reference transcriptome. For example, this entails single sample transformations like a non-linearly transformation by e.g. robust spline normalization toward the reference expression profile or require parameter assessment (e.g. mean and standard deviation per gene) per batch (i.e. a collection of sample expression values obtained from samples that underwent a comparable processing in terms of sample storage and workup, reagents, processing times, etc.). These batch normalization parameters must be determined based on data comparable to the reference expression profile (i.e. a demographic and clinically homogeneous population of sufficient size e.g., suffering from NDMM), such that batch correction can be applied to any future sample (including e.g., non-NDMM) that underwent comparable processing. For example, batch specific means and standard deviations per gene are shifted and scaled respectively toward the mean and standard deviations per gene as observed in the reference transcriptome.

For example, the expression levels of the marker set in a sample are normalized to indicate an increase or decrease of the expression of the markers in the marker set. The expression profile in a sample constituting from the individual expression levels of the single two or more markers, is for example compared to the reference expression profile of the marker set to determine whether the subject expression profile is sufficiently similar to the reference profile.

Alternatively, the expression profile of the sample is compared to a more than one reference expression profiles to select the reference expression profile that is most similar to the subject expression profile.

The reference expression profile is for example a predetermined expression profile. Alternatively the expression profile of a reference is determined when determining the marker set expression profile in the sample. The reference expression profile is for example the average of the expression profiles in a particular group of samples, such as a group of disease samples. For example, the reference expression profile is the average of the expression profiles in a group of rare disease samples or samples of high grading of a disease.

For example, for normalization purposes, the reference expression profile is a demographic (e.g. gender, age, race, etc.) and clinically (e.g. chromosomal aberrations, disease grade, etc.) homogeneous population of n>50 for which the mean expression and its standard deviation per gene are characteristic. For example, the reference expression profile is a demographic and clinically homogeneous population of n between 50 to 500, n between 75 to 400, n between 100 to 350, n between 150 to 300, or n between 200 to 250. For example, the reference expression profile is a demographic and clinically homogeneous population of n=154.

Any method known in the art for comparing two or more data sets to detect similarity between them may be used to compare the expression profile of the sample to the reference expression profiles.

In machine learning and statistics, classification refers to identifying to which set of categories a new observation belongs, on the basis of a training set of data containing observations (or instances) whose category membership is known. An algorithm that implements classification, especially in a concrete implementation, is known as a classifier. Many classifiers are known in the art, with linear or non-linear classifier boundaries, such as but not limited to: ClaNC, nearest mean classifier, weighted voting method, simple Bayes classifier, linear discriminant analysis (LDA), quadratic discriminant analysis (QDA), Support Vector Machines (SVM), or the k-nearest neighbor (k-nn) classifier.

The PCL-like transcriptomic status determined by the marker set of the present invention represents an expression profile of a sample. A PCL-like transcriptomic status determined by the marker set of the present invention is for example indicative for a disease if the expression profile is similar to the expression profile of a sufficiently large reference group of disease samples, based on a score that is equal or larger than the lowest scoring disease sample in this reference group, to a score equal or larger than the highest disease sample.

In one example the PCL-like transcriptomic status is an expression profile of a sample that is similar to the transcriptomic profiles of a sufficiently large reference cohort of pPCL bone marrow tumor samples, based on a score that is equal or larger than the lowest scoring pPCL sample in this reference cohort, to a score equal or larger than the highest score of 10% of the lowest scoring pPCL samples in this reference cohort.

The score is for example calculated by computing the first principal component from the expression profile of the marker set. For example the score is calculated by computing the first principal component of the expression profile of the marker set in the classifier's discovery data. In one example, the classifier's discovery data is obtained from a demographically (e.g. gender, age, race, etc.) and clinically (e.g. chromosomal aberrations, disease grade, etc.) homogeneous population of n=109 NDMM patients. Any means for calculating the first principal component may be used. For example, principal components are determined using the “prcomp” function in R package “stats” (version 4.0.2) according to R Core Team REfSC: R: A Language and Environment for Statistical Computing, 2020. It is within the purview of the skilled person to obtain a suitable sample for determining a PCL-like transcriptomic status by the marker set of the present invention. For example, the sample is selected from plasma cell, blood, (pre-) malignant plasma cell, bone marrow, urine, serum, cells and tissue, such as tumor tissue or tumor cells, or a combination thereof.

### Methods According to the Present Invention

The present invention likewise refers to a method for determining a PCL-like transcriptomic status indicative for a disease in a sample comprising the steps of
a) isolating RNA from the sample
b) determining the expression profile of the marker set according to the present invention in the isolated RNA,
c) calculating a score, wherein the score is based on the first principal component of the expression profile of the marker set in a classifier's discovery data, and
d) comparing the score calculated in step c) to a reference score.

Isolation of RNA may be performed by any suitable method known in the art and as described herein, respectively.

For example total RNA of sufficient quality and quantify is isolated from a tumor. RNA quantification is performed, and values are normalized to obtain read-outs which are compatible with the classifiers discovery setting.

The marker set for determining the expression profile in a sample is chosen as described herein. For example, the method comprises determining the expression profile of all markers of Table 5.

The expression levels of the single markers as well as the expression profile of the marker set is determined by any means of the art, e.g., as described herein.

For example, total RNA is isolated from the sample. RNA quantity and quality are assessed. Tumor cells optionally comprise ≥80% of the cells in the sample as assessed by flow cytometry (or ≥90% morphologically) and a Bioanalyzer RNA integrity number ≥7.

Quantification of the RNA can be performed on any platform (e.g. microarray, NGS RNASeq, qRT-PCR, etc.), e.g., if a kit is used according to the manufacturer's instructions. The first normalization steps is for example performed according to the manufacturer's instructions. Quantifications is for example summarized in terms of the Ensembl v74 gene model, and expressed on log 2 scale (e.g. log 2 intensity for microarray, log 2 (TPM+1) for RNASeq, or ΔCt for qRT-PCR).

Depending on the platform used, additional corrections and normalization are required as described herein.

Calculating the score which is based on the first principal component of the expression profile of the marker set will be performed as described herein.

Further, the method includes comparing the calculated score to a reference score. The reference score is for example based on the expression profile of a reference as described herein. For example, the reference score is based on the first principal component of the expression levels of the marker set of the present invention in the reference. The reference varies and is selected depending on the score to be determined.

For example, the reference score is predetermined or determined in parallel to the determination of the score in a sample. Alternatively, the reference score is a generally established score which is indicative for a disease.

A reference is used for comparison and classification of the measurements and analysis obtained by the present invention. For example, reference refers to pPCL (e.g. when determining the reference score), or reference refers to PCL and NDMM (e.g. when calculating the principal components), or reference refers to NDMM (e.g. in case of the normalization steps).

The calculated score of the expression profile determines the PCL-like transcriptomic status of the sample indicating a disease. For example, the reference score is the lowest score that 100% of the samples in a reference have a higher score. For example, the reference score is the lowest score that at least 60%, at least 65%, at least 70%, at least 75%, at least 80% at least 85%, at least 90% at least 95% at least 95% of the samples in a reference have a higher score. For example, the reference score is the lowest score that at least 70 to 90%, at least 75 to 95% at least 80 to 97%, at least 85 to 99%, or at least 90 to 100% of the samples in a reference have a higher score.

For example, the score which indicates a disease corresponding to the disease of the reference is in the range of at least 1 to 7. For example, the score which indicates a disease corresponding to the disease of the reference is in the range of at least 0.1 to 15, of at least 0.3 to 15, of at least 0.5 to 15, of at least 1 to 10, of at least 1.5 to 8, of at least 2 to 7, of at least 2.5 to 5 or of at least 3 to 7, or a combination thereof. For example, the score which indicates a disease corresponding to the disease of the reference is at least 0.1, at least 0.3, at least 0.5, at least 0.7, at least 1.0, at least 1.5, at least 2.0, at least 2.5, at least 3.0, at least 3.5, at least 4.0, at least 4.5. In one example, the score which indicates a disease corresponding to a pPCL reference is at least 3.55.

The method of the present invention optionally further comprises determining a CTC level in a sample. The CTC level in a sample is for example determined by quantification according to any suitable method of the art. For example, the CTC level in a sample is quantified using flow cytometry (e.g., FACS), VDJ sequencing, morphologically, or using immunocapture technologies. For example, the CTC level is quantified as described in the following examples using flow cytometry. For example, the CTC level is quantified as described in the following examples using Next Generation Flow (NGF).

Further, the method optionally comprises determining the tumor burden. Tumor burden is for example generally be determined based on the percentage of plasma cells in bone marrow, M-protein in serum and/or urine, the level of beta-2 microglobulin in serum, the level of lactate dehydrogenase in serum or by imaging.

The expression profile of the marker set in a sample is for example referenced to the CTC level in a sample. For example, the combination of the expression profile in a sample and the CTC level for example strengthens the indication of a disease by the method of the present invention.

A CTC level indicating a rare disease or a high grade of a disease in the sample is for example between 0.001 to 100%, 0.01 to 100%, 0.1 to 100%, 1 to 100%, or 5 to 100%. For example, the CTC level in the sample is 0.001 to 99%, 0.01 to 98%, 0.05 to 97%, 0.1 to 96%, 0.5 to 95%, 1 to 94%, 3 to 93%, 5 to 92%, 7 to 92%, 9 to 90%, 10 to 85%, 12 to 80%, 15 to 75%, 17 to 70%, 18 to 65%, 19 to 60%, 20 to 55%, 22 to 50%, 25 to 45%, 30 to 55%, 35 to 60%, or 40 to 80%. Further, the CTC level indicating a rare disease or a high grade disease in the sample is for example ≥5%, ≥7%, ≥10%, ≥12%, ≥15%, ≥17% or ≥20%. In an example, a CTC level ≥2×10⁹/L is indicating pPCL.

Determining the CTC level in a sample having a marker set expression profile that corresponds to the marker set expression profile of a reference having an increased CTC level, e.g., of 5 to 30%, facilitates discrimination between two diseases or grades of a diseases. Moreover, a CTC level serves as a threshold allowing differentiation between two diseases or grades of a disease. For example, a CTC level of at least 5% or 20% is used as a threshold. In one example, a CTC level of ≥5% in a sample indicates pPCL, wherein a CTC level in a sample of <5% indicates NDMM. Alternatively, a CTC level of ≥20% in a sample indicates pPCL, wherein a CTC level in a sample of <20% indicates PCL-like MM.

Optionally, the CTC level in a sample is referenced to the tumor burden. Referencing the CTC level to the tumor burden for example further strengthens the validity of the CTC level in a sample. For example, lower CTC levels in the sample of a subject, e.g. a CTC level of <5%, is associated with a lower tumor burden in the subject. On the contrary, higher CTC levels are for example associated with a higher tumor burden.

Referencing the expression profile in a sample to the CTC level referenced to the tumor burden for example further strengthens the indication of a disease by the method of the present invention.

Optionally, the expression profile is referenced to the molecular profile (i.e., mutational, copy number and cytogenetic profile) of the sample. Optionally the expression profile is referenced to the epigenetic profile (for instance the methylome) of the sample. Determining the molecular profile and the epigenetic profile is performed according to the standard methods known to a person skilled in the art. It is clear to the skilled person that the expression profile is for example referenced to one or more selected from the group consisting of CTC level, tumor burden, molecular profile, epigenetic profile, or a combination thereof.

The marker set and the method of the present invention enables detection of diseases, such as rare diseases and/or high-grading diseases which are hardly or not or at least nor reliably detectable by any method of the state of the art. Once the disease is detected by the present invention, it's severity is optionally double checked by at least one prognostic risk model known in the art for the specific disease.

A prognostic risk model grades the disease progression and defines the state on a disease. A prognostic risk model optionally provides information about disease progression, survival, treatment response or a combination thereof in a subject.

For example, prognostic risk models for plasma cell dyscrasias like MM comprise R-ISS, ISS, FISH, SKY92, UAMS70, Durie-Salmon Staging etc.

Both, the International Staging System (ISS) and the revised International Staging System (R-ISS) have been developed by the International Myeloma Working Group (IMWG), providing a prognostic risk model based on the serum β2 microglobulin (Sβ2M) value and serum albumin value in a subject. For the R-ISS two additional prognostic factors have been incorporated which are the risk of chromosomal abnormalities (CA) as assessed by fluorescence in-situ hybridization (FISH) and the serum level of lactate dehydrogenase level (LDH).

ISS:

| Stage | Values (β2M = Serum β2 microglobulin; ALB = serum albumin) |
|---|---|
| I | β2M < 3.5 mg/L; ALB ≥ 3.5 g/dL |
| II | β2M < 3.5 mg/L; ALB < 3.5 g/dL; or β2M 3.5-5.5 mg/L |
| III | β2M > 5.5 mg/L |

R-ISS:

| Stage | Criteria |
|---|---|
| I | Serum β2 microglobulin < 3.5 mg/l |
| | Serum albumin ≥ 3.5 g/dl |
| | Standard-risk chromosomal abnormalities (CA) |
| | Normal LDH |
| II | Not R-ISS stage I or III |
| III | Serum β2 microglobulin ≥ 5.5 mg/L and either |
| | High-risk CA by FISH |
| | OR |
| | High LDH |

FISH is used to screen for chromosomal abnormalities and allows cytogenetic risk stratification of myeloma. Subjects are considered to have high-risk disease if FISH studies demonstrate for example one of the following chromosomal abnormalities: t (14;16), t (4;14), or loss of p53 gene locus (del (17p) or monosomy 17).

For example, the method according to the present invention further comprises determining the grade of a disease according to at least one prognostic risk model as described above. For example, the at least one prognostic risk model is selected from the group consisting of R-ISS status, ISS status, FISH status, SKY92 status, UAMS70 status or a combination thereof.

In particular, methods are provided for classifying, determining a treatment, or determining the prognosis of an individual, said method comprising determining a PCL-like transcriptomic status from a sample from said individual, as disclosed herein, and determining the SKY92 risk status from the sample. The SKY92 risk status may be determined by measuring the expression levels of the markers in Table 7 and classifying the individual as having a high or standard SKY92 risk status based on said expression levels. As exemplified in FIG. 11D, individuals can thus be classified as 1) PCL-like/SKY92 standard risk, 2) PCL-like/SKY92 high risk, 3) not PCL-like (e.g., i-MM)/SKY92 standard risk, and 4) not PCL-like (e.g., i-MM)/SKY92 high risk. As discussed further below, the classification of an individual based on the PCL-like transcriptomic status and SKY92 risk status can be used when selecting appropriate treatment.

Further, the present invention comprises the selection of a treatment of a disease in a subject in need thereof based on the PCL-like transcriptomic status. For example, the marker set or the method of the present invention is used for selecting a therapy to prevent and/or treat a disease in a subject in need thereof. For example, the marker set or the method of the present invention is used for selecting an active agent for preventing and/or treating a disease in a subject in need thereof.

For example, based on the PCL-like transcriptomic status determined according to the present invention, a cancer treatment is selected. For example, based on the PCL-like transcriptomic status determined according to the present invention an active agent such as an “adjuvant treatment” is selected. Adjuvant treatment, as used herein, refers to the administration of one or more drugs to a patient after surgical resection of one or more cancerous tumors, where all resectable disease (i.e. cancer) has been removed from the patient, but where there remains a statistical risk of relapse. Adjuvant treatment is useful to diminish the likelihood or the severity of reoccurrence or the disease.

Active agents are for example selected from the group consisting of a chemotherapeutic, targeted therapy drugs, immunotherapy drugs, an antagonist modulating the expression of one or more genes of the marker set of the present invention, or a combination thereof.

For example, the active agent is selected from the group consisting of monoclonal antibodies (e.g., daratumumab (Darzalex), elotuzumab (Empliciti)), BCL-2 inhibitors (e.g., venetoclax (Venclexta), navitoclax), selinexor, PRC2 inhibitors, nucleoside analogs, dacarbazine (DTIC), temozolomide (Temodal), carboplatin (Paraplatin, Paraplatin AQ), paclitaxel (Taxol), cisplatin (Platinol AQ), andvinblastine and (Velbe), BRAF inhibitors (vemurafenib (Zelboraf) and dabrafenib (Tafinlar)) and MEK inhibitors (cobimetinib (Cotellic) and trametinib (Mekinist)), BTK inhibitors, cytokines (e.g., Interferon alfa-2b or Interleukin-2) immune checkpoint inhibitors (e.g., Ipilimumab (Yervoy), Nivolumab (Opdivo), Pembrolizumab (Keytruda)), proteasome inhibitors (e.g., bortezomib (Velcade), carfilzomib (Kyprolis), ixazomib (Ninlaro)), immunomodulators (e.g., thalidomide, lenalidomide (Revlimid), pomalidomide), CAR-T cells, bispecific antibodies, NK cell therapy, autologous stem cell transplantation, allogenic stem cell transplantation, radiation therapy, oncolytic immunotherapy, or a combination thereof.

Individuals classified as having a SKY92 high risk status are preferably treated more aggressively (e.g., quadruplet induction therapy including anti-CD38 and high dose autologous stem cell transplantation therapy), and better patient monitoring, than individuals having a SKY92 standard risk status. Individuals classified as having a PCL-like transcriptomic status should receive more aggressive treatment (e.g., quadruplet induction therapy including anti-CD38 and high dose autologous stem cell transplantation therapy), and better patient monitoring than individuals that do not have a PCL-like transcriptomic status. Individuals classified as having a SKY92 high risk status and a PCL-like transcriptomic status are treated more aggressively than individuals without a PCL-like transcriptomic status and a SKY92 low risk status. Aggressive treatment comprises for example quadruplet induction therapy including anti-CD38 and high dose autologous stem cell transplantation therapy, and better patient monitoring. Additional therapy for patients with PCL high risk profile comprises for example experimental treatment with bispecific antibodies and CAR-T cell approaches.

Within the research field of multiple myeloma, trial designs have started to focus on high-risk disease specifically, i.e., it has become particularly relevant to perform adequate diagnostic assessments at baseline to screen patients for inclusion in these kind of trials (e.g., the MUKnine OPTIMUM trial (NCT03188172)). High-risk status defined by the present invention is for example used as an inclusion criterium for risk-adapted trials.

Further, it is helpful to know high-risk status at baseline for example to enable clinicians to better monitor the patients during and after treatment, as these patients may suffer from highly proliferative progressive disease (PD). To allow for an earlier treatment start of aggressive PD, these patients could benefit from more intensified follow-up protocols with for instance Minimal Residual Disease (MRD) assessments with Next Generation Flow (NGF) or Next Generation Sequencing (NGS) approaches.

Suitable active agents are for example administered by any appropriate route. Suitable routes include oral, rectal, nasal, topical (including buccal and sublingual), vaginal, and parenteral (including subcutaneous, intramuscular, intravenous, intradermal, intrathecal, and epidural).

For example, the marker set or the method of the present invention is used in the field of personalized medicine for individually treatment of a subject in need thereof.

### Kits of the Present Invention

The present invention is further directed to a kit for determining a PCL-like transcriptomic status which is indicative for a disease. The kit comprises or consists of means for determining the expression profile of the marker set of the present invention in a sample. Such means facilitate specific detection and/or binding to the one or more genes comprised by the marker set of the present invention. For example, such means are required for performing qRT-PCR, gene sequencing, microarrays etc.

It is well within the purview of the skilled person to identify and develop such means facilitating specific binding to the marker set of the present invention. For example, such means comprise reagents, probes, primers, proteins, peptides, antibodies, antibody fragments, antigens etc.

In some embodiments the kits comprise primer pairs or probes specific for the marker sets described herein. In some embodiments the kits comprise primer pairs or probes for housekeeping genes. In some embodiments, the kits further comprising one or more of the following: DNA polymerase, deoxynucleoside triphosphates, buffer, and Mg²⁺. In some embodiments, the kits comprise a control nucleic acid for one or more, preferably for each, primer pair. Preferably, the control nucleic acid is cDNA and more preferably the cDNA corresponds to a sequence that spans at least one intron/exon boundary of the respective gene. Such cDNA is useful to distinguish gene expression from genomic contamination. In some embodiments, one or more primers of the primer pair are chemically modified. Such modified primers include fluorescently or radioactively labeled primers.

Optionally, the kit further comprises means for determining the CTC level in a sample. For example, the kit comprises means for performing flow cytometric measurements.

Optionally, the kit further comprises means for determining the tumor burden in a sample. For example, the kit comprises means for performing flow cytometric measurements.

Identification and development means for determining the CTC level in a sample and means for determining tumor burden is performed according to the standard methods known to a person skilled in the art.

Optionally, the kit further comprises means for determining the grade of a disease according to prognostic risk model, such as R-ISS status, ISS status, FISH status, SKY92 status UAMS70 status, TP53 mutational status or a combination thereof.

Identification and development means for determining the grade of a disease according to a prognostic risk model is performed according to the standard methods known to a person skilled in the art. Such means for example comprise probes, primers, reagents, dyes, fluorescent probes, proteins, peptides, antibodies etc.

The kit as described herein, optionally further comprises an active agent for use in method of preventing and/or treating a disease, for example a rare disease or a high grade of a disease.

Optionally the kit of the present invention further comprise instructions for use of the kit and/or interpretation of the measurements obtained by the kit. Moreover, the kit comprises for example suitable references and reference scores, respectively.

In addition or alternatively, the kit of the present invention further comprises for example means for sample collection, sample processing, sample storage, product insert, or combinations thereof.

A subject and/or patient of the present invention is for example a mammalian such as a human, cat, dog or horse, a bird or a fish.

### EXAMPLES

### A: Experimental Design

This study consisted of two main phases:

Construction and validation of a molecular classifier for plasma cell leukemia-like (PCL-like) disease (cohort 1):
The PCL-like classifier was constructed in a discovery cohort consisting of newly diagnosed multiple myeloma (NDMM) and primary PCL (pPCL) samples (discovery cohort).

The PCL-like classifier was validated in a separate cohort consisting of NDMM and pPCL samples (validation cohort).

Assessment of the prevalence and prognostic value of a classifier for PCL-like disease (cohort 2):

Additional datasets together with the discovery and validation cohort were leveraged to assess the prevalence of PCL-like transcriptomic status in a range of CD138-enriched plasma cell samples. These included healthy plasma cells, monoclonal gammopathy of undetermined significance (MGUS), smoldering MM (SMM), NDMM, pPCL, circulating tumor cell (CTC) and cell line samples (prevalence cohort).

The association of PCL-like transcriptomic status with progression-free survival (PFS) and overall survival (OS) was assessed in both univariate, meta-analysis and multivariate models in a subset of patients from the prevalence cohort, comprising a total of seven NDMM cohorts, which were independent of the discovery and validation cohort (survival cohort).

### B: Patient Selection

All human investigations in this study were performed after approval by medical ethical committees. All patients included in this study have provided written informed consent, in concordance with the Declaration of Helsinki.

### Discovery and Validation Cohort

In this cohort, patients from the Cassiopeia trial (NCT02541383) (n=171) were included, who had been enrolled in a hospital in Belgium or the Netherlands, as well as patients from the EMN12/HO129 (EudraCT 2013-005157-75) (n=51) and HO143 trials (EudraCT 2016-002600-90) (n=126), of whom baseline CTC levels had been quantified (see, Moreau P. et al., Lancet 394:29-38, 2019; Zweegman S. et al., Blood 134:695-695, 2019; Musto P. et al., Blood 134:693-693, 2019). A subset of patients with transcriptomic data of their bone marrow (BM) tumor cells was selected for either the discovery (n=124) or validation phase (n=59) of the PCL-like classifier (unpublished tumor transcriptomic profiles; deposited under accession numbers GSE164701, GSE164830 and GSE164703).

### Prevalence Cohort

In this cohort, all patients with available tumor transcriptomics from the discovery and validation sets were included, as well as patients with unpublished tumor transcriptomic profiles from the EMN02/HO95 trial (unpublished tumor transcriptomic profiles; deposited under accession number GSE164706) and 7 previously published datasets with transcriptomic data from plasma cells.

N=22 healthy plasma cell samples, n=44 MGUS and n=12 SMM CEL files were downloaded from the Gene Expression Omnibus (GEO) (GSE5900), as well as n=328 HOVON-65/GMMG-HD4 (GSE19784), n=180 HOVON-87/NMSG-18 (GSE87900), n=247 MRC-IX (GSE15695), n=345 Total Therapy 2 (GSE24080), n=214 Total Therapy 3 (GSE24080) and n=4 MM cell line (GSE159289) CEL files. NDMM patients from the HOVON-65/GMMG-HD4, HOVON-87/NMSG-18, EMN02/HO95, MRC-IX, Total Therapy 2 and Total Therapy 3 were included in subsequent analyses if a baseline tumor sample had been obtained from BM and if data on both patient age, progression-free survival (PFS) and overall survival (OS) were available.

For a subset of EMN02/HO95 NDMM samples (n=123), tumor transcriptomic data from both microarray and RNA Seq data were generated (unpublished tumor transcriptomic profiles; deposited under accession number GSE164847). Paired microarray and RNA Seq data were used to compare classifier scores between platforms, whereas only microarray data of these patients were used in all other analyses.

Data from patients enrolled in the HOVON-65/GMMG-HD4, HOVON-87/NMSG-18, EMN02/HO95, Cassiopeia and EMN12/HO129 trials were used for the comparison of baseline data between intramedullary (i-MM), PCL-like MM and pPCL patients, as a comparable set of baseline characteristics was available from these trial cohorts. The same cohort was used for comparison of ssGSEA scores between i-MM, PCL-like MM and pPCL tumor samples.

Transcriptomic data from CTCs were obtained from patients from the EMN12/H0129 cohort. For comparisons of scores between BM and CTC samples from pPCL patients, only pre-treatment samples were used.

MM cell lines in our dataset were represented by transcriptomic profiles from the OPM-2, EJM, MOLP-8 and JJN-3 cell lines (see Katagiri S. et al., Int J Cancer 36:241-6, 1985; Hamilton M S. et al, 1990; Matsuo Y. et al., Leuk Res 28:869-77, 2004; Jackson N. et al, Clin Exp Immunol 75:93-9, 1989).

### Survival Cohort

This cohort consisted of all NDMM patients from the prevalence cohort, who had been included in the HOVON-65/GMMG-HD4 (EudraCT 2004-000944-26), HOVON-87/NMSG-18 (EudraCT 2007-004007-34), EMN02/HO95 (EudraCT 2009-017903-28), MRC-IX (ISRCTN68454111), Total Therapy 2 (NCT00083551) and Total Therapy 3 (A: NCT00081939, B: NCT00572169) studies. Please refer to the respective study publications and/or trial registers for a detailed description on patient eligibility criteria and used treatment protocols, which have been summarized in Table 3 (see Sonneveld P. et al, J Clin Oncol 30:2946-55, 2012; Zweegman S. et al, Blood 127:1109-16, 2016; Cavo M. et al, Lancet Haematol 7: e456-e468, 2020; Morgan G. J. et al, Blood 118:1231-8, 2011; Morgan G. J. et al, Haematologica 97:442-50, 2012; Barlogie B. et al, Int J Hematol 76 Suppl 1:337-9, 2002; Barlogie B. et al, Br J Haematol 138:176-85, 2007).

### C: Sample Processing

Only sample processing procedures for samples from in the discovery, validation and EMN02/HO95 prevalence cohorts are discussed. Please refer to the original publications for additional information on specific sample processing procedures for all other cohorts (see Zhan F. et al, Blood 109:1692-700, 2007; Broyl A. et al, Blood 116:2543-53, 2010; Kuiper R. et al, Blood Adv 4:6298-6309, 2020; Dickens N. J. et al, Clin Cancer Res 16:1856-64, 2010; Zhan F. et al, Blood 108:2020-8, 2006; van Beers E. H. et al, J Mol Diagn 23:120-129, 2021).

### Tumor Samples

Before treatment start, a BM aspirate sample was collected for all NDMM patients, whereas both a BM and peripheral blood (PB) sample were obtained from pPCL patients. Samples were shipped to Erasmus MC, Rotterdam, the Netherlands by overnight express courier and de-identified upon receipt. Tumor cell enrichment was generally performed within 36 hours after sampling, by means of CD138 positive cell selection with the EasySep™ Human Whole Blood and Bone Marrow CD138 Positive Selection Kit II (STEMCELL Technologies, catalog number 17887RF) on the mononuclear cell fraction. After tumor cell enrichment, aliquots of generally 1×106 cells were lysed in 600 μL RLT Plus buffer (Qiagen, catalog number 1053393), snap frozen in liquid nitrogen and stored at −80° C.

### Tumor Purity Assessment

Of all enriched tumor samples in this study, purity was assessed after CD138 positive cell selection. Purity assessment was performed by both flow cytometry and morphology for each sample, with morphological purity assessment alone being performed if cell numbers were limited.

For morphological purity assessment, one cytospin was generated of a single cell suspension of 33×10³ cells, followed by a May-Grünwald-Giemsa staining. Per slide, 100-200 intact cells were evaluated by a specialist in hemato-cytology. Purity assessment by flow cytometry was performed on a FACSCanto II (BD) machine. To this end, 1×10⁵ cells were stained with a staining panel including CD138-PE (Beckman Coulter, catalog number A54190), CD38-PE-Cy7 (BD, catalog number 335825), CD45-APC (BD, catalog number 555485), annexin-FITC (Tau Technologies, catalog number A700) and DAPI (Thermo Fisher Scientific, catalog number D3571). Flow cytometric sample purity was defined as the percentage of CD45−/dimCD38+/++ events within a population of DAPI-leukocytes.

### RNA Isolation and Quality Checks

Total RNA was isolated with the AllPrep DNA/RNA Mini Kit (Qiagen, catalog number 80204). RNA quantity and quality were assessed on a NanoDrop 3300 fluorometer (ThermoFisher Scientific), whereas the RNA integrity number (RIN) was measured on a Bioanalyzer 2100 machine (Agilent) with the RNA 6000 Nano Kit (Agilent, catalog number 5067-1511).

### Tumor Sample Selection

Tumor samples were selected for subsequent transcriptomic profiling if these had a tumor purity of ≥80% as assessed by flow cytometry (or ≥90% morphological purity, in case no flow cytometric purity assessment had been performed) and a RIN ≥7. Additional quality criteria that were applied for microarray samples have been published previously (see Kuiper R. et al, Blood Adv 4:6298-6309, 2020).

### Microarray

Microarray data were generated on the MMprofiler™ (SkylineDx), for which Human Genome U133 Plus 2.0 Arrays (Affymetrix) were used. Arrays were processed as described in detail previously (see van Beers E. H. et al, J Mol Diagn 23:120-129, 2021).

### RNA Seq Library Preparation and Sequencing

RNA Seq libraries were generated with the mRNA HyperPrep Kit (KAPA, catalog number 08105952001/KK8544), according to manufacturer's instructions. In short, 250 ng of total RNA was used for poly(A) selection, after which magnesium-based fragmentation was conducted. A median fragment length of 200-300 bp was aimed for, using a fragmentation time of 6 minutes at 94° C. After cDNA synthesis and A-tailing, custom adapters were ligated (Integrated DNA Technologies), followed by 11 cycles of library amplification. The quality of the generated libraries was assessed on the Bioanalyzer 2100 machine (Agilent) with the High Sensitivity DNA kit (Agilent Technologies, catalog number 5067-4626). Libraries were quantified on a 7500 Fast Real-Time PCR System (Applied Biosystems) machine using the NEBNext Library Quant Kit for Illumina (New England BioLabs, catalog number #E7630S/L).

Paired-end sequencing of libraries was performed on a NovaSeq 6000 (Illumina) machine, with a read length of 2×10¹ bp and an average of 55×106 reads per sample (see Table 1).

TABLE 1
Quality metrics RNA Seq data

| | Number | Number of | |
|---|---|---|---|
| | of reads | aligned reads | Percentage of |
| Sample ID | (×10\hat{ }6) | (×10\hat{ }6) | aligned reads |
| H143_11405259490_v1 | 58.8 | 47.3 | 80.5 |
| H143_12250278397_v1 | 50.5 | 42.6 | 84.4 |
| H143_15899371102_v1 | 54.3 | 44 | 81.1 |
| H143_17006911740_v1 | 56.7 | 47 | 82.9 |
| H143_17226505219_v1 | 58.1 | 49 | 84.4 |
| H143_19913049186_v1 | 62.5 | 52.9 | 84.7 |
| H143_22685180872_v1 | 75.1 | 52.7 | 70.2 |
| H143_23906322534_v1 | 53.4 | 40.7 | 76.2 |
| H143_27424011493_v1 | 56.3 | 46.4 | 82.5 |
| H143_29580873118_v1 | 39.4 | 32.8 | 83.3 |
| H143_30826692821_v1 | 56.3 | 45 | 79.8 |
| H143_32025485018_v1 | 43.6 | 34.7 | 79.5 |
| H143_34194087698_v1 | 49.7 | 38.4 | 77.2 |
| H143_34221056807_v1 | 53.7 | 44.8 | 83.5 |
| H143_35481538763_v1 | 57.4 | 45.5 | 79.2 |
| H143_36215534375_v1 | 55.8 | 45.2 | 80.9 |
| H143_36676992427_v1 | 64.3 | 51.8 | 80.5 |
| H143_37852117733_v1 | 53.7 | 42.3 | 78.9 |
| H143_42664338057_v1 | 56.7 | 45.4 | 80 |
| H143_43886189351_v1 | 61.4 | 48.4 | 78.8 |
| H143_49921048448_v1 | 48 | 39.2 | 81.6 |
| H143_50271695959_v1 | 70.7 | 56.3 | 79.6 |
| H143_59458239686_v1 | 72.4 | 59.9 | 82.8 |
| H143_59902067724_v1 | 52.1 | 41.5 | 79.6 |
| H143_60925049292_v1 | 60.9 | 50.1 | 82.3 |
| H143_61936532503_v1 | 49.4 | 39.9 | 80.7 |
| H143_62146312806_v1 | 65.3 | 53.7 | 82.3 |
| H143_67136338923_v1 | 66.7 | 55.2 | 82.8 |
| H143_68173853894_v1 | 63 | 51.8 | 82.2 |
| H143_69254957465_v1 | 44.3 | 35.7 | 80.7 |
| H143_69507292376_v1 | 47.2 | 41 | 86.8 |
| H143_73592528004_v1 | 60.7 | 48.3 | 79.6 |
| H143_75736348582_v1 | 72.7 | 57.6 | 79.2 |
| H143_78086106590_v1 | 65.5 | 52.3 | 79.9 |
| H143_84330021249_v1 | 51.3 | 41.4 | 80.7 |
| H143_87595859584_v1 | 59.3 | 47.1 | 79.4 |
| H143_89726683769_v1 | 68.1 | 57.1 | 83.8 |
| H143_90232462705_v1 | 64 | 52.1 | 81.3 |
| H143_93624264385_v1 | 70 | 54.5 | 77.8 |
| H143_94102396117_v1 | 48.3 | 39.1 | 81.1 |
| H143_95512868312_v1 | 41.8 | 33.6 | 80.3 |
| H143_96755058147_v1 | 56.8 | 46.4 | 81.6 |
| H143_97284639677_v1 | 47.3 | 37.3 | 78.7 |
| H143_99804551870_v1 | 76.8 | 63.7 | 82.9 |
| H143_99912777286_v1 | 79.4 | 64.9 | 81.8 |
| H95_14482288896_v1_NGS | 59.6 | 47.4 | 79.6 |
| H95_16722157346_v1_NGS | 58.4 | 51.7 | 88.5 |
| H95_16923322959_v1_NGS | 59.4 | 52.3 | 88 |
| H95_17121435841_v1_NGS | 41.2 | 34.8 | 84.4 |
| H95_17665771071_v1_NGS | 41.3 | 34.2 | 82.8 |
| H95_18039603324_v1_NGS | 60.4 | 51.9 | 86.1 |
| H95_19105755822_v1_NGS | 137 | 120.4 | 87.9 |
| H95_20532640307_v1_NGS | 35.9 | 28.9 | 80.4 |
| H95_21787494486_v1_NGS | 45.6 | 38.6 | 84.7 |
| H95_22514675883_v1_NGS | 44.7 | 39.1 | 87.5 |
| H95_25158603699_v1_NGS | 60.7 | 53 | 87.2 |
| H95_25293719664_v1_NGS | 56.4 | 47.3 | 83.9 |
| H95_25459089354_v1_NGS | 50.4 | 44.3 | 87.9 |
| H95_25778709384_v1_NGS | 65.2 | 52.1 | 79.9 |
| H95_26506671996_v1_NGS | 51.9 | 44.7 | 86.1 |
| H95_27178908090_v1_NGS | 48 | 36.2 | 75.5 |
| H95_27201492856_v1_NGS | 45.7 | 37 | 81 |
| H95_27225006672_v1_NGS | 55.6 | 48.5 | 87.2 |
| H95_27354837176_v1_NGS | 46.3 | 36.2 | 78.2 |
| H95_28035359704_v1_NGS | 49.3 | 42.8 | 86.7 |
| H95_28299419011_v1_NGS | 58.7 | 48.3 | 82.3 |
| H95_28487116855_v1_NGS | 54.1 | 48.2 | 89 |
| H95_29140118011_v1_NGS | 61.2 | 50.6 | 82.8 |
| H95_30114194799_v1_NGS | 51.1 | 42.4 | 83.1 |
| H95_30406789186_v1_NGS | 45.8 | 41 | 89.4 |
| H95_30933175219_v1_NGS | 53.2 | 45.3 | 85.2 |
| H95_31765020247_v1_NGS | 48.9 | 43.7 | 89.3 |
| H95_32313700572_v1_NGS | 46.1 | 41.3 | 89.5 |
| H95_32948916566_v1_NGS | 57.1 | 49.5 | 86.7 |
| H95_33103500276_v1_NGS | 47.4 | 39.4 | 83.1 |
| H95_33221184711_v1_NGS | 43 | 34.4 | 80 |
| H95_33350375676_v1_NGS | 50.6 | 44.7 | 88.4 |
| H95_33374926438_v1_NGS | 48.5 | 40.6 | 83.6 |
| H95_34136296948_v1_NGS | 65.9 | 52 | 78.8 |
| H95_35062128235_v1_NGS | 53 | 42.3 | 79.8 |
| H95_35677547599_v1_NGS | 54.4 | 47 | 86.5 |
| H95_36160911021_v1_NGS | 49.4 | 38.6 | 78.1 |
| H95_36204590648_v1_NGS | 61.9 | 52.5 | 84.8 |
| H95_37926530551_v1_NGS | 56.3 | 49.6 | 88.1 |
| H95_38004909435_v1_NGS | 40.2 | 34.3 | 85.2 |
| H95_40001226014_v1_NGS | 55.2 | 45.9 | 83.2 |
| H95_40862784553_v1_NGS | 50 | 41 | 82 |
| H95_41639388983_v1_NGS | 41.9 | 34.4 | 82.1 |
| H95_41822734775_v1_NGS | 61.5 | 52.8 | 85.9 |
| H95_42265336143_v1_NGS | 43.6 | 35.9 | 82.3 |
| H95_42724425741_v1_NGS | 46.6 | 39.6 | 85 |
| H95_42826179782_v1_NGS | 48.6 | 42.1 | 86.5 |
| H95_43011810355_v1_NGS | 45.5 | 37.9 | 83.4 |
| H95_43027278927_v1_NGS | 62.7 | 54.4 | 86.7 |
| H95_43459586815_v1_NGS | 67.2 | 53.7 | 79.9 |
| H95_43919813020_v1_NGS | 44.3 | 35 | 78.9 |
| H95_44330907233_v1_NGS | 82.6 | 66.8 | 80.9 |
| H95_44393599343_v1_NGS | 53.2 | 44.7 | 84 |
| H95_44472878275_v1_NGS | 61.6 | 49.9 | 81 |
| H95_44945778546_v1_NGS | 41.9 | 34.3 | 81.8 |
| H95_45211526870_v1_NGS | 39.7 | 33.9 | 85.4 |
| H95_45295560206_v1_NGS | 43.1 | 37.5 | 87 |
| H95_45671714120_v1_NGS | 54.3 | 44.7 | 82.2 |
| H95_46043575096_v1_NGS | 71.9 | 61.9 | 86.1 |
| H95_46241617256_v1_NGS | 62.5 | 51.6 | 82.5 |
| H95_46490107996_v1_NGS | 47.6 | 39.2 | 82.4 |
| H95_47363361239_v1_NGS | 55.5 | 49.7 | 89.6 |
| H95_48538742214_v1_NGS | 68.1 | 57.6 | 84.6 |
| H95_49394530211_v1_NGS | 66.5 | 59.7 | 89.8 |
| H95_49394683789_v1_NGS | 67.3 | 56.1 | 83.3 |
| H95_51074988620_v1_NGS | 47.8 | 39.3 | 82.3 |
| H95_51513539224_v1_NGS | 50 | 41.3 | 82.5 |
| H95_54073444830_v1_NGS | 55.4 | 46.7 | 84.4 |
| H95_54317412108_v1_NGS | 46.9 | 38.8 | 82.7 |
| H95_54622906537_v1_NGS | 71.2 | 61.6 | 86.4 |
| H95_55663140178_v1_NGS | 59.7 | 51.6 | 86.5 |
| H95_57558990134_v1_NGS | 40.4 | 33.4 | 82.7 |
| H95_59287418008_v1_NGS | 54.8 | 46.1 | 84 |
| H95_60297585008_v1_NGS | 44.7 | 35 | 78.3 |
| H95_60725268705_v1_NGS | 53.8 | 44.5 | 82.8 |
| H95_61064339613_v1_NGS | 48.2 | 42.3 | 87.7 |
| H95_62341947243_v1_NGS | 53.5 | 46.6 | 87.1 |
| H95_63502541661_v1_NGS | 56.1 | 46.3 | 82.4 |
| H95_63571029752_v1_NGS | 63.6 | 49 | 77.1 |
| H95_63575013733_v1_NGS | 44.7 | 37.3 | 83.4 |
| H95_63759484796_v1_NGS | 46.7 | 41.3 | 88.3 |
| H95_64091355837_v1_NGS | 53.6 | 44.1 | 82.3 |
| H95_65014493851_v1_NGS | 54.6 | 42.4 | 77.6 |
| H95_66242972109_v1_NGS | 60.4 | 49.9 | 82.6 |
| H95_67510998658_v1_NGS | 58.4 | 48.2 | 82.6 |
| H95_68092466641_v1_NGS | 56.3 | 48.6 | 86.4 |
| H95_68829024685_v1_NGS | 62.4 | 52.6 | 84.2 |
| H95_68858331506_v1_NGS | 62.1 | 53.4 | 86.1 |
| H95_70289119654_v1_NGS | 68.9 | 54.5 | 79.1 |
| H95_70590289754_v1_NGS | 42.3 | 36.8 | 86.9 |
| H95_70997597735_v1_NGS | 53.6 | 47.5 | 88.6 |
| H95_71035164901_v1_NGS | 77.4 | 64 | 82.7 |
| H95_72661535288_v1_NGS | 57.6 | 48.6 | 84.3 |
| H95_72699532466_v1_NGS | 63.1 | 50.3 | 79.6 |
| H95_73535534583_v1_NGS | 57.1 | 48.4 | 84.6 |
| H95_73938687550_v1_NGS | 47.2 | 41.1 | 87.1 |
| H95_75107900786_v1_NGS | 8.4 | 6.8 | 80.4 |
| H95_75486530924_v1_NGS | 47.2 | 39.4 | 83.4 |
| H95_75510431627_v1_NGS | 57.2 | 46.5 | 81.3 |
| H95_76299194805_v1_NGS | 45.4 | 35.9 | 79.1 |
| H95_79093593121_v1_NGS | 74.5 | 62.6 | 84 |
| H95_79099048168_v1_NGS | 51.9 | 41.7 | 80.4 |
| H95_79165043271_v1_NGS | 58.5 | 48.3 | 82.5 |
| H95_80428083070_v1_NGS | 62 | 52.5 | 84.8 |
| H95_81233173985_v1_NGS | 57.6 | 47.9 | 83.2 |
| H95_81588441726_v1_NGS | 56.1 | 46.2 | 82.4 |
| H95_82069194919_v1_NGS | 65.1 | 56.9 | 87.3 |
| H95_82543072604_v1_NGS | 48.2 | 39.5 | 82 |
| H95_86492226861_v1_NGS | 50.1 | 42.7 | 85.2 |
| H95_86616095024_v1_NGS | 49.9 | 40.4 | 80.9 |
| H95_88467538747_v1_NGS | 61.6 | 53.8 | 87.5 |
| H95_88927351997_v1_NGS | 53.9 | 46.3 | 85.8 |
| H95_89034225809_v1_NGS | 48.6 | 42.3 | 87.1 |
| H95_89673138286_v1_NGS | 45.2 | 39.8 | 88.1 |
| H95_93029417313_v1_NGS | 47.7 | 39.6 | 83 |
| H95_93466300400_v1_NGS | 45.4 | 39.1 | 86.2 |
| H95_93707430173_v1_NGS | 40.5 | 34.2 | 84.5 |
| H95_94551085868_v1_NGS | 59.2 | 51.5 | 87 |
| H95_95268571667_v1_NGS | 51 | 44.1 | 86.5 |
| H95_95730729146_v1_NGS | 50.5 | 41.1 | 81.3 |
| H95_97651650339_v1_NGS | 40 | 34.2 | 85.6 |
| H95_98855783526_v1_NGS | 57.4 | 49.6 | 86.4 |
| H95_99118191515_v1_NGS | 50.5 | 42.2 | 83.6 |

### CTC Level Quantification

Baseline CTC levels were quantified for patients enrolled in the Cassiopeia, HO143 and EMN12/HO129 trials. For all NDMM patients, CTC levels were quantified by flow cytometry. To this end, 6-10 mL of PB was drawn before treatment start and shipped to Erasmus MC, Rotterdam, the Netherlands, by overnight express courier and de-identified upon receipt. Samples were processed and analyzed according to standardized Next Generation Flow (NGF) methods (EuroFlow) (see Flores-Montero J. et al, Leukemia 31:2094-2103, 2017; Hofste op Bruinink D. et al, Haematologica 106:1496-1499, 2020)

In short, ≤36 hours after sampling NH4Cl bulk lysis was performed. Subsequently, the sample was divided over two tubes (100 μL with 106 cells each) and stained according to the EuroFlow NGF protocol, using CD138, CD38, CD45, CD19, CD27 and CD56 as backbone markers, with CD81 and CD117 as additional markers for tube 1, and CyIgL in combination with CyIgK as additional markers for tube 2. Cells were measured on either a FACSCanto™ II (BD) or FACSLyric™ (BD) machine, using EuroFlow settings (see Kalina T. et al, Leukemia 26:1986-2010, 2012; Glier H. et al, J Immunol Methods 475:112680, 2019). Data analysis was performed in Infinicyt (version 2.0, Cytognos). A total of ≥5×106 leukocytes was aimed to be acquired per tube. A population of ≥20 monoclonal plasma cells (mPCs) was required for CTC identification, which translated into a theoretical limit of detection (LOD) of 20/1×10⁷=2×10⁻⁶ per CTC assay (see Arroz M. et al, Cytometry B Clin Cytom 90:31-9, 2016; Paiva B. et al, J Clin Oncol 38:784-792, 2020). The percentage of CTCs was defined as the number of mPCs/the number of leukocytes x 100.

For all pPCL patients, CTCs were detected and quantified at baseline by routine morphological assessment of blood smears in local hematology laboratories, after which data were collected and curated by the EMN data center. A subset of NDMM and pPCL patients had their baseline CTC levels quantified by both NGF and morphological assessment. For all subsequent CTC level analyses, NGF CTC levels were used for all NDMM patients, whereas morphological CTC levels were used for all pPCL patients.

### CTC Immunophenotyping

Samples in which ≥150 CTCs had been quantified by flow cytometry were used for immunophenotypic characterization. A marker was defined as positive if ≥10% of mPCs had a EuroFlow-standardized staining intensity of >10³ (arbitrary fluorescent units). Markers that were positive or negative in all samples were excluded in correlative analyses.

### Cytogenetics

Cytogenetic aberrations were assessed by interphase fluorescence in situ hybridization (FISH) on CD138-enriched, chemotherapy-naive plasma cells, according to technical quality criteria that have been established within the framework of the European Myeloma Network (EMN) (see Ross F. M. et al, Haematologica 97:1272-7, 2012). Translocations of the immunoglobulin heavy chain (IgH) were detected with probes for t (4;14) (FGFR3/WHSC1), t (8;14) (MYC), t (11;14) (CCND1) and t (14;16) (MAF), whereas copy number aberrations involving deletion of chromosome 1p32 (del1p32) (CDKN2C), 13q14 (del13q14) (RB1) and 17p13 (del17p13) (TP53), as well as gain of chromosome 1q21 (gain1q21) (CKS1B) and hyperdiploidy were detected with either interphase FISH or high-density SNP arrays.

High-risk FISH status was defined according to criteria from the International Myeloma Working Group (IMWG) and included the presence of either a t (4;14), t (14;16) and/or del17p13.28 The presence of a primary IgH translocation was defined as having either a t (4;14), t (11;14) or t (14;16). Patients that had been tested positive for one primary IgH translocation were classified as negative for the other two primary IgH translocations. Hyperdiploidy was defined as having >2 gains of chromosomes 5, 9, 11, 15. Non-hyperdiploid status was defined as having no gains in ≥3 chromosomes out of chromosomes 5, 9, 11, 15. All reported prevalences were calculated based on the following formula: the number of patients with the respective cytogenetic aberrancy/the number of tested patients*100%.

The detection of cytogenetic aberrations in the remaining datasets of this study have been described in detail elsewhere (see Morgan G. J. et al, Blood 118:1231-8, 2011; Barlogie B. et al, Int J Hematol 76 Suppl 1:337-9, 2002; Barlogie B. et al, Br J Haematol 138:176-85, 2007; Kuiper R. et al, Blood Adv 4:6298-6309, 2020; Neben K., et al, Blood 119:940-8, 2012).

### D: Bioinformatic Pipeline

### Data Preprocessing—Microarray Data

For all datasets, the mas5 function of R package “affy” (version 1.63.0) was applied to run a background correction, scale the arrays towards a mean expression value of 500 and summarize features into Ensembl gene IDs using brain array (version 18) ENSG CDF (see Gautier L. et al, Bioinformatics 20:307-15, 2004; Dai M. et al, Nucleic Acids Res 33: e175, 2005). Gene expression values were transformed into a log 2 intensity scale.

### Data Preprocessing—RNA Seq Data

Fastq files were constructed using “bcl2Fastq” (version 2.20.0.422, Illumina), after which universal adapters were removed using “Trim galore” (version 0.4.4) (https://github.com/FelixKrueger/TrimGalore). Transcript per million (TPM) counts were measured on the trimmed Fastq files using Salmon (version 1.3.0) with an adapted version of the Ensembl (release 74) reference transcriptome, which has been described in the document “MMRF_COMMpass_IA15_Methods.pdf” (MMRF Researcher Gateway, https://research.themmrf.org) (see Patro R. et al, Nat Methods 14:417-419, 2017; Hubbard T. et al, Nucleic Acids Res 30:38-41, 2002). Transcripts were summarized into gene level TPM values using R package “tximport” (version 1.14.2).

Thereafter, both sets were merged, mitochondrial genes and ribosomal proteins were excluded and TPM was recalculated accounting for all remaining transcripts, excluding IgH-related genes (see Soneson C. et al, F1000Res 4:1521, 2015). All gene expression values were subsequently transformed into a log 2 (TPM+1) intensity scale.

### Batch Correction

To account for nonlinear global differences between platforms, a robust spline normalization was applied towards the Cassiopeia microarray samples using the “rsn” function in R package “lumi” (version 2.42.0) (see Du P. et al, Bioinformatics 24:1547-8, 2008). In RNA Seq samples, only expressed genes (i.e. TPM>0) were taken into account. Subsequently, a 2D UMAP dimension reduction analysis for the top 30 principal components was performed, using R package “umap” (version 0.2.7.0) to identify distinct batches closely corresponding with technical variation (see McInnes L., et al, arXiv.org, 2020). To specifically account for major batch effects, gene centric mean/variance normalization was performed towards the NDMM samples in the discovery cohort, using the batch-specific NDMM samples derived from BM. All expressed genes (i.e. log 2 expression >5 in >75% of samples in the discovery cohort) were subsequently used as input for all further downstream analyses.

### Data Analysis

### Principal Component Analysis

Principal components were determined using the “prcomp” function in R package “stats” (version 4.0.2) (see R Core Team REfSC, 2020). Input expression values were centered, but not scaled to unit variance.

### Construction and Validation of the Classifier

The PCL-like classifier was trained on data from patients in the discovery cohort, who presented with a CTC level ≥LOD and who had matched tumor transcriptomics, tumor burden and CTC level data.

The training phase consisted of three steps. First, genes were identified that associated with CTC levels (percentage of CTCs), independent of tumor burden (percentage of plasma cells in the bone marrow aspirate). To this end the linear regression model y=β₀+β₁x₁+β₂x₂+∈ was applied using R package “limma” (version 3.46.0) (see Ritchie M. E. et al, Nucleic Acids Res 43: e47, 2015). In this model, y represents the logit-transformed CTC level, x₁ the logit-transformed tumor burden, for which the baseline percentage of plasma cells in the BM aspirate was used, x₂ the expression of the gene of interest on log 2 scale, β the regression estimates and ∈ the modeling error. CTC-associated genes with a false discovery rate (FDR)<0.05 were considered significant.

The second step was aimed at identifying the number of CTC-associated genes with which pPCL could be best distinguished from NDMM samples. To this end, a leave-one-out cross validation analysis was performed. In this analysis, each fold consisted of all samples in the discovery cohort minus one that was left out. Per fold, step one of the training phase was repeated, obtaining a ranking of all genes based on the significance of the association with CTC levels, independent of tumor load. Subsequently, the first principal component (PC1) was determined, for each combination of an increasing number of genes that were most significantly associated with CTC levels, ranging from 20 to 1000 genes, thereby rotating PC1 such that it positively correlated with CTC levels. Subsequently, a projection was computed for the sample that had been left out. This resulted in a specific cross-validated PC1 score for each pPCL and NDMM sample in this analysis, for each classifier size. The optimal number of genes for the PCL-like classifier was defined as the lowest possible number of genes with which the highest discriminative power was achieved to distinguish pPCL and NDMM samples, using a Wilcoxon test. Thereafter, the score was calculated by computing the first principal component from the expression values of this optimal number of genes, using all samples in the discovery cohort as input. The obtained loadings per PCL-like classifier gene were subsequently used to calculate the score for all remaining samples in cohort 2.

In the third step of training the PCL-like classifier, a cutoff was determined. Hereto, the lowest score was selected with which all pPCL samples in the discovery cohort could be identified.

The PCL-like classifier was validated in an independent validation cohort by means of two analyses:
The proportion of PCL patients that are correctly identified by the classifier (i.e. sensitivity)
The percentage of variance in predicted CTC was determined that could be explained by a combination of the score and tumor burden. To this end, the correlation coefficient was computed of the linear regression model y=β₀+β₁x₁+β₂x₃+∈, using the variables as described above and the score x₃.

For the first analysis, all samples in the validation cohort were used, whereas for the second analysis only matched CTC level, tumor burden and tumor transcriptomics data were used from patients with a CTC level ≥LOD.

### Other Gene Classifiers and MM Clusters

The MMprofiler™ gene expression assay (SkylineDx) was used to determine the SKY92 high-risk classification and MM clusters in microarray samples (see Broyl A. et al, Blood 116:2543-53, 2010; Kuiper R. et al, Leukemia 26:2406-13, 2012).

SKY92 (=EMC92) scores were calculated as described in Kuiper et al 2012. Briefly, the SKY92 is a summation of the weighted expression of 92 probe sets (see Table 7). This signature constitutes a linear model, expressed in the following formula:

$$SKY 92 ( x ) = {\sum }_{i = 1}^{92} {β}_{i} {x}_{i}$$

where Bi represents the weight factor of gene i, and xᵢ represents the expression level of gene i in a patient. Based on their SKY92 score, patients were split into two groups, those above the threshold of 0.7774 were classified as positive (High Risk), and those below the threshold as negative (Standard Risk).

Positive beta values (i.e., weight values) indicate that increased expression of said gene over a reference value indicates a positive contribution towards the SKY92 score, as a consequence a larger chance of being above the threshold. Conversely, positive beta values indicate that decreased expression of said gene over a reference value indicates a negative, contribution towards the SKY92 score.

Negative beta values indicate that decreased expression of said gene over a reference value indicates a positive contribution towards the SKY92 score, as a consequence a larger chance of being above the threshold. Conversely, negative beta values indicate that increased expression of said gene over a reference value indicates a negative, contribution towards the SKY92 score.

The following Table 2 shows SKY92 probe sets and weights:

| Probesets | Beta | Gene Symbol |
|---|---|---|
| 200701_at | −0.0210 | NPC2 |
| 200775_s_at | 0.0163 | HNRNPK///MIR7-1 |
| 200875_s_at | 0.0437 | MIR1292///NOP56///SNORD110/// |
| | | SNORD57///SNORD86 |
| 200933_x_at | −0.0323 | RPS4X |
| 201102_s_at | 0.0349 | PFKL |
| 201292_at | −0.0372 | TOP2A |
| 201307_at | 0.0165 | SEPTIN11/SEPT11 |
| 201398_s_at | −0.0254 | TRAM1 |
| 201555_at | −0.0052 | MCM3 |
| 201795_at | 0.0067 | LBR |
| 201930_at | −0.0090 | MCM6 |
| 202107_s_at | 0.0225 | MCM2 |
| 202322_s_at | 0.0129 | GGPS1 |
| 202532_s_at | −0.0006 | DHFR |
| 202542_s_at | 0.0870 | AIMP1 |
| 202553_s_at | 0.0054 | SYF2 |
| 202728_s_at | −0.1105 | LTBP1 |
| 202813_at | 0.0548 | TARBP1 |
| 202842_s_at | −0.0626 | DNAJB9 |
| 202884_s_at | 0.0714 | PPP2R1B |
| 203145_at | −0.0002 | SPAG5 |
| 204026_s_at | 0.0046 | ZWINT |
| 204379_s_at | 0.0594 | FGFR3 |
| 205046_at | 0.0087 | CENPE |
| 206204_at | 0.0477 | GRB14 |
| 207618_s_at | 0.0746 | BCS1L |
| 208232_x_at | −0.0493 | NRG1 |
| 208667_s_at | −0.0390 | ST13 |
| 208732_at | −0.0618 | RAB2A |
| 208747_s_at | −0.0874 | C1S |
| 208904_s_at | −0.0334 | RPS28 |
| 208942_s_at | −0.0997 | SEC62 |
| 208967_s_at | 0.0113 | AK2 |
| 209026_x_at | 0.0255 | TUBB |
| 209683_at | −0.0561 | CYRIA (*FAM49A) |
| 210334_x_at | 0.0175 | BIRC5 |
| 211714_x_at | 0.0221 | TUBB |
| 211963_s_at | 0.0303 | ARPC5 |
| 212055_at | 0.0384 | TPGS2 |
| 212282_at | 0.0530 | TMEM97 |
| 212788_x_at | −0.0164 | FTL |
| 213002_at | −0.0418 | MARCKS |
| 213007_at | −0.0106 | FANCI |
| 213350_at | 0.0056 | RPS11 |
| 214150_x_at | −0.0349 | ATP6V0E1 |
| 214482_at | 0.0861 | ZBTB25 |
| 214612_x_at | 0.0496 | MAGEA6 |
| 215177_s_at | −0.0768 | ITGA6 |
| 215181_at | −0.0342 | CDH22 |
| 216473_x_at | −0.0576 | DUX2///DUX4///DUX4L2/// |
| | | DUX4L3///DUX4L4///DUX4L5/// |
| | | DUX4L6///DUX4L7/// |
| | | LOC100288627///LOC100288657/// |
| | | LOC652119 |
| 217548_at | −0.0423 | ARPIN (LOC100129502/C15orf38) |
| 217728_at | 0.0773 | S100A6 |
| 217732_s_at | −0.0252 | ITM2B |
| 217824_at | −0.0041 | UBE2J1 |
| 217852_s_at | 0.0008 | ARL8B |
| 218355_at | 0.0116 | KIF4A |
| 218365_s_at | 0.0035 | DARS2 |
| 218662_s_at | −0.0176 | NCAPG |
| 219510_at | −0.0097 | POLQ |
| 219550_at | 0.0559 | ROBO3 |
| 220351_at | 0.0420 | ACKR4 (*CCRL1) |
| 221041_s_at | −0.0520 | SLC17A5 |
| 221606_s_at | 0.0208 | HMGN5 |
| 221677_s_at | 0.0126 | DONSON |
| 221755_at | 0.0396 | EHBP1L1 |
| 221826_at | 0.0200 | ANGEL2 |
| 222154_s_at | 0.0154 | SPATS2L |
| 222680_s_at | 0.0205 | DTL |
| 222713_s_at | 0.0278 | FANCF |
| 223381_at | −0.0070 | NUF2 |
| 223811_s_at | 0.0556 | GET4///SUN1 |
| 224009_x_at | −0.0520 | DHRS9 |
| 225366_at | 0.0140 | PGM2 |
| 225601_at | 0.0750 | HMGB3 |
| 226217_at | −0.0319 | SLC30A7 |
| 226218_at | −0.0644 | IL7R |
| 226742_at | −0.0345 | SAR1B |
| 228416_at | −0.0778 | ACVR2A |
| 230034_x_at | −0.0330 | MRPL41 |
| 231210_at | 0.0093 | MAJIN (*C11orf85) |
| 231738_at | 0.0686 | PCDHB7 |
| 231989_s_at | 0.0730 | 61E3.4///LOC100132247/// |
| | | LOC100271836///LOC100652992/// |
| | | LOC613037///LOC728888/// |
| | | NPIPL3///SLC7A5P1///SMG1P1 |
| 233399_x_at | −0.0184 | TMED10P1///ZNF252 |
| 233437_at | 0.0446 | GABRA4 |
| 238116_at | 0.0661 | DYNLRB2 |
| 238662_at | 0.0490 | ATPBD4 |
| 238780_s_at | −0.0529 | KCNJ5 |
| 239054_at | −0.1088 | SFMBT1 |
| 242180_at | −0.0585 | TSPAN16 |
| 243018_at | 0.0407 | BBOX1-AS1 |
| 38158_at | 0.0423 | ESPL1 |
| AFFX-HUMISGF3A/ | 0.0525 | STAT1///STAT1 |
| M97935_MA_at | | |

*Gene annotation updated from Kuiper et al. 2012

MM clusters were subsequently merged into a CD1/CD2 cluster (comprising clusters CD1 and CD2) and non-IgH cluster (comprising clusters HY, PR, CTA, LB, NFKB, NP, myeloid and PRL3), resulting in four main clusters: CD1/CD2, MF, MS and non-IgH. The UAMS70 high-risk classification was calculated as described in the original publication (see Shaughnessy J. D. et al, Blood 109:2276-84, 2007).

### Conversion of Gene Classifiers for RNA Seq Data

Microarray-developed gene classifiers were converted for RNA Seq datasets according to a bioinformatic pipeline that has been outlined in detail previously (see Kuiper R. et al, Blood Adv 4:6298-6309, 2020). To check the validity of this procedure, paired PCL-like, SKY92 and UAMS70 scores were generated from samples with both array and RNA Seq transcriptomic data. Scores were compared in a linear regression model, using the “Im” function in R package “stats” (version 4.0.2) (see R Core Team REfSC, 2020).

Single sample gene set enrichment analysis Single sample gene set enrichment analysis (ssGSEA) was performed on tumor transcriptomic data from all HOVON-65/GMMG-HD4, HOVON-87/NMSG-18, EMN02/HO95, Cassiopeia and EMN12/HO129 microarray samples in the prevalence cohort, using an in-house written R package that computationally optimized the publicly available ssGSEA GenePattern module (https://github.com/GSEA-MSigDB/ssGSEA-gpmodule) (see Barbie D. A. et al, Nature 462:108-12, 2009; Subramanian A. et al, Proc Natl Acad Sci USA 102:15545-50, 2005). Gene sets from the curated canonical pathways MSigDB Collections (c2.cp, version 7.1) were selected for subsequent analyses if these had ≥10 genes overlap with expressed genes in the discovery cohort.

### E: Survival Analysis

### Cox Regression Analysis

Univariate and multivariate survival analyses were performed with a Cox regression model using R package “survival” (version 3.2.3), for which baseline and follow-up data from the survival cohort were used.⁴³ Follow-up time was measured from start of treatment to either the occurrence of an event or last contact in case of no event. For PFS, an event was defined as either progressive disease or death from any cause. For OS, an event was defined as death from any cause. All multivariate survival analyses were stratified by trial cohort and included age≤65 years as covariate.

### Meta-Analysis

Meta-analyses were performed using R package “meta” (version 4.15.1), using a random effects model (see Balduzzi S. et al, Evid Based Ment Health 22:153-160, 2019). The Mantel-Haenszel formula was used to pool study cohort data, with between study variance being estimated with the DerSimonian and Laird procedure. Test statistics and confidence intervals were adjusted with the Hartung and Knapp method.

### F: Data Visualization

Figures were generated in RStudio (version 1.4.1103), with R packages “ggplot2” (version 3.3.2), “ggExtra” (version 0.9), “corrplot” (version 0.84), “ggridges” (version 0.5.2), “pheatmap” (version 1.0.12), “viridis” (version 0.5.1), “meta” (version 4.15-1) and “survminer” (version 0.4.8), as well as in Adobe Illustrator (version 25.1, Adobe) (see Balduzzi S. et al, Evid Based Ment Health 22:153-160, 2019; RStudio Team R, 2016; Wickham H., Springer-Verlag New York, 2016; Attali D. et al, R package version 0.9, 2019; Wei T. et al, R package “corrplot” 2017; Wilke C. O., R package version 0.5.2, 2020; Kolde R., R package version 1.0.12, 2019; Garnier S., R package version 0.5.1. 2018; Kassambara A. et al, R package version 0.4.8. 2020).

### G: Data Management

Baseline and follow-up data of all NDMM and pPCL patients in this study were systematically collected and curated in the context of nine registered clinical trials (Table 2). Baseline and follow-up data for the HOVON-65/GMMG-HD4, HOVON-87/NMSG-18 and HO143 trials were provided by Hemato-Oncology Foundation for Adults in the Netherlands (HOVON), for the Cassiopeia trial by the Intergroupe Francophone du Myelome (IFM) and for the EMN02/HO95 and EMN12/HO129 trials by EMN. For patients enrolled in the Total Therapy 2 and 3 protocols, these data were obtained from GEO (GSE24080), whereas clinical data from MRC IX trial patients were kindly shared by Dr. Walter Gregory.

### H: Data Availability

Salmon TPM count data from the EMN02/H095 and HO143 cohorts, as well as CEL files from the EMN02/H095, EMN12/HO129 and Cassiopeia cohorts are available on the GEO repository (https://www.ncbi.nlm.nih.gov/geo/), under accession codes GSE164847, GSE164830, GSE164706, GSE164703 and GSE164701, respectively (see Table 2).

TABLE 3
Treatment protocols and inclusion criteria per trial cohort
NDMM datasets

| | | Main | | | [symbol] | | | | Intensification 1 | | |
|---|---|---|---|---|---|---|---|---|---|---|---|
| | Registration | inclusion | Study | | Alternative | Alternative | Alternative | Alternative | Alternative | Alternative | Alternative |
| Test | number | criteria | type | Randomization | 1 | 2 | 3 | 4 | 1 | 2 | 3 |
| HOVON-65/GMMG-HD4 | [symbol] 2004- [symbol] | Transplant- | Phase II | Upfront, between | [symbol] | [symbol] | | | [symbol] | | |
| | | eligible NDMM, | trial | arm A and arm E. | | | | | | | |
| | | age 18-65 | | | | | | | | | |
| HOVON-87-NMSG-18 | [symbol] 2007- [symbol] | NDMM, age >85 | Phase II | Upfront, between | [symbol] | [symbol] | | | | | |
| | | or age ≤85 and | trial | arm A and arm B. | | | | | | | |
| | | transplant- | | | | | | | | | |
| | | ineligible | | | | | | | | | |
| EMN02-HO95 | [symbol] 2018- [symbol] | Transplant- | Phase II | Randomization 1: | [symbol] | | | | [symbol] | [symbol] | [symbol] |
| | | eligible NDMM, | trial | after induction | | | | | | | |
| | | age 18-65 | | between arm 1A, | | | | | | | |
| | | | | 1B, and 1C. | | | | | | | |
| | | | | Randomization 2: | | | | | | | |
| | | | | after | | | | | | | |
| | | | | consolidation, | | | | | | | |
| | | | | between arm 2A | | | | | | | |
| | | | | and 2B. | | | | | | | |
| Cassiopeia | NCT02541385 | Transplant- | Phase II | Randomization 1: | [symbol] | [symbol] | | | [symbol] | | |
| | | eligible NDMM, | trial | upfront, between | | | | | | | |
| | | age 18-65 | | arm 1A and 1B. | | | | | | | |
| | | | | Randomization 2: | | | | | | | |
| | | | | after | | | | | | | |
| | | | | consolidation, | | | | | | | |
| | | | | between arm 2A | | | | | | | |
| | | | | and 2B. | | | | | | | |
| HO143 | [symbol] 2018- [symbol] | Transplant- | Phase I | No randomization | [symbol] | | | | | | |
| | | ineligible NDMM, | trial | | | | | | | | |
| | | age ≥18, until | | | | | | | | | |
| | | end trial | | | | | | | | | |
| MRC-4X | [symbol] | NDMM, age ≥18; | Phase II | Intensive pathway | [symbol] | [symbol] | [symbol] | [symbol] | [symbol] | | |
| | | pathway | trial | Randomization 1: | | | | | | | |
| | | selection based | | upfront between | | | | | | | |
| | | on performance | | arm 1A, 1B, 1C, | | | | | | | |
| | | status, [symbol] | | and 1D. | | | | | | | |
| | | judgement | | Randomization 2: | | | | | | | |
| | | and [symbol] | | after | | | | | | | |
| | | | | intensification, | | | | | | | |
| | | | | between arm 2A, | | | | | | | |
| | | | | 2B, 2C, and 2D. | | | | | | | |
| | | | | Non-intensive | [symbol] | [symbol] | [symbol] | [symbol] | | | |
| | | | | pathway | | | | | | | |
| | | | | Randomization 1: | | | | | | | |
| | | | | upfront, between | | | | | | | |
| | | | | arm 1A, 1B, 1C, | | | | | | | |
| | | | | and 1D | | | | | | | |
| | | | | Randomization 2: | | | | | | | |
| | | | | after induction, | | | | | | | |
| | | | | between arm 2A, | | | | | | | |
| | | | | 2B, 2C, and 2D. | | | | | | | |
| Total Therapy 2 | NCT0083551 | NDMM, age | Phase II | Randomization 1: | [symbol] | [symbol] | | | [symbol] | [symbol] | |
| | | 18-75 years | trial | Upfront between | | | | | | | |
| | | | | arm A and arm B; | | | | | | | |
| | | | | Randomization 2: | | | | | | | |
| | | | | after | | | | | | | |
| | | | | intensification | | | | | | | |
| | | | | between arm a and | | | | | | | |
| | | | | b, which was | | | | | | | |
| | | | | modified into | | | | | | | |
| | | | | one consolidation | | | | | | | |
| | | | | treatment [symbol] | | | | | | | |
| | | | | after [symbol] of | | | | | | | |
| | | | | 121 patients. | | | | | | | |
| Total Therapy 3 | NCT [symbol] , | NDMM, age | Phase I | No randomization | [symbol] | | | | [symbol] | | |
| (A + B) | NCT00572189 | 18-75 years | trial | | | | | | | | |
| MMRF CoMMpass | NCT01484297 | [symbol] | [symbol] | [symbol] | | | | | | | |

| | | |
|---|---|---|
| Consolidation | Intensification 2 | Maintenance |

| Alternative 2 | Alternative 2 | Alternative 3 | Alternative 4 | Alternative 5 | Alternative 6 | Alternative 1 | Alternative 2 | Alternative 1 | Alternative 2 | Alternative 3 | Alternative 4 |
|---|---|---|---|---|---|---|---|---|---|---|---|
| | | | | | | | | [symbol] | [symbol] | | |
| | | | | | | | | [symbol] | [symbol] | | |
| [symbol] | [symbol] | | | | | | | [symbol] | | | |
| [symbol] | [symbol] | | | | | | | [symbol] | [symbol] | | |
| | | | | | | | | [symbol] | | | |
| | | | | | | | | [symbol] | [symbol] | [symbol] | [symbol] |
| | | | | | | | | [symbol] | [symbol] | [symbol] | [symbol] |
| [symbol] | [symbol] | [symbol] | [symbol] | [symbol] | [symbol] | | | [symbol] | [symbol] | | |
| [symbol] | | | | | | | | [symbol] | | | |

pPCL

[symbol]

| | | | | |
|---|---|---|---|---|
| Registration | Main inclusion | Study | [symbol] | Intensification 1 |

| Dataset | number | criteria | type | Randomization | Alternative 1 | Alternative 2 | Alternative 3 | Alternative 4 | Alternative 1 | Alternative 2 | Alternative 3 |
|---|---|---|---|---|---|---|---|---|---|---|---|
| [symbol] | [symbol] | [symbol] -eligible | [symbol] | No randomization, | [symbol] | [symbol] | | | [symbol] | [symbol] | [symbol] |
| | | pPCL 18- | | treatment | | | | | | | |
| | | [symbol] years | | allocation over | | | | | | | |
| | | | | [symbol] A, B and C [symbol] | | | | | | | |
| | | [symbol] pPCL [symbol] | | No [symbol] | [symbol] | | | | | | |
| | | [symbol] years | | | | | | | | | |

| | | |
|---|---|---|
| Consideration | Intensification 2 | Maintenance |

| Alternative 1 | Alternative 2 | Alternative 3 | Alternative 4 | Alternative 5 | Alternative 6 | Alternative 1 | Alternative 2 | Alternative 1 | Alternative 2 | Alternative 3 | Alternative 4 |
|---|---|---|---|---|---|---|---|---|---|---|---|
| [symbol] | [symbol] | [symbol] | | | | [symbol] | [symbol] | [symbol] | [symbol] | | |
| | | | | | | | | [symbol] | | | |

[symbol]

indicates data missing or illegible when filed

### Example 1: Baseline Characteristics of pPCL Versus NDMM

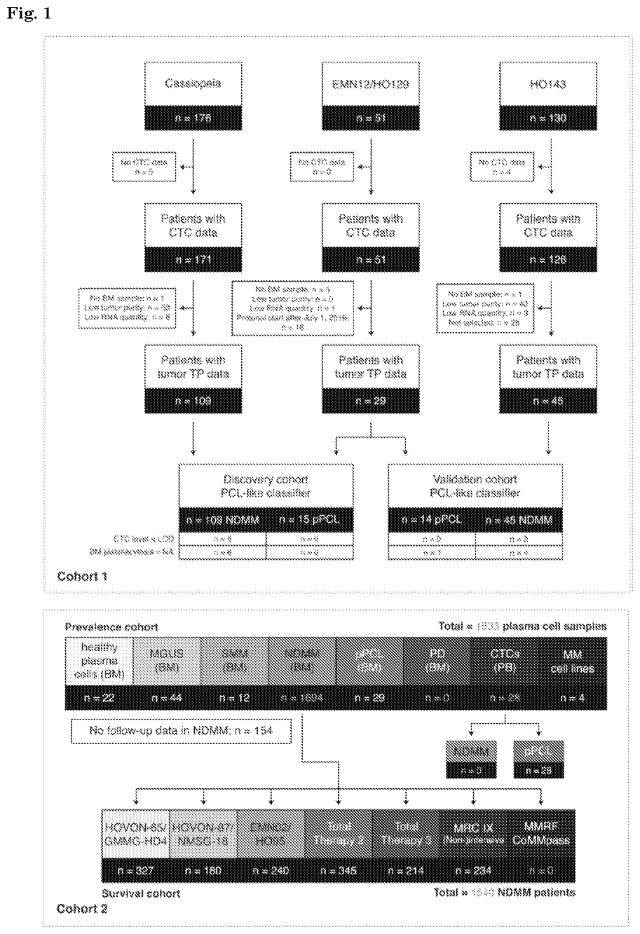

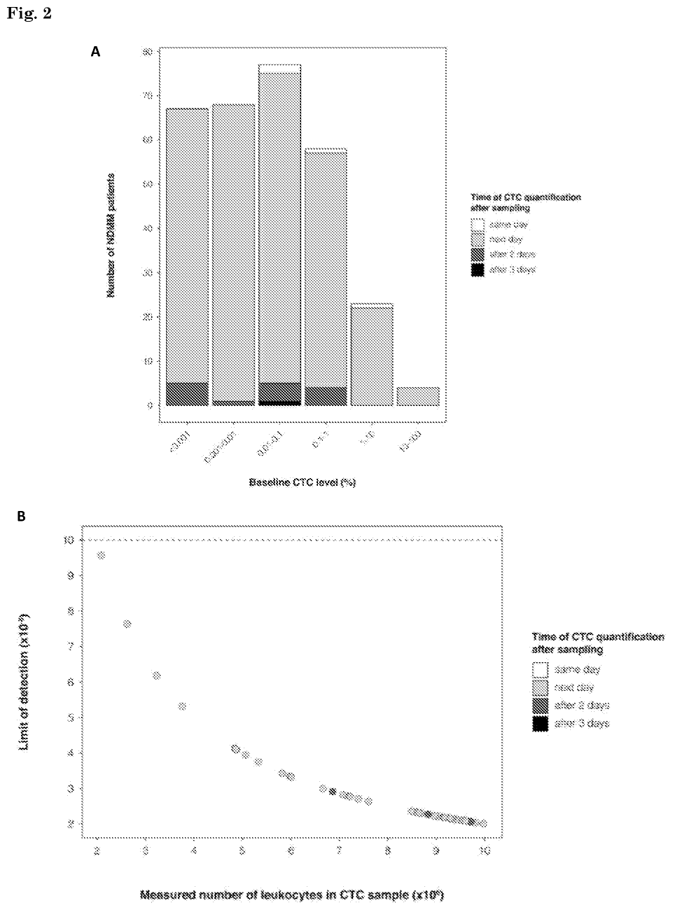

To investigate clinical and molecular determinants of PCL-like disease, baseline patient and tumor characteristics were collected of 297 NDMM and 51 pPCL patients (cohort 1) (FIG. 1, Table 4). NGF was performed to quantify CTCs in NDMM patients, which could be detected in 257/297 (87%) patients (range, 0.00028%-36%), with 40/40 (100%) CTC-negative assays reaching a limit of detection <10-5 (FIG. 2).

TABLE 4
Baseline characteristics of patients with CTC level data per trial cohort

| Trial | | | |
|---|---|---|---|
| EMN12/HO129 | Cassiopeia | HO143 | |
| (pPCL)* | (NDMM)** | (NDMM) | Overall |
| Total number of patients in trial | | | |
| 51 | 176 | 130 | 357 |
| Patients with baseline CTC level data (%) | | | |
| 51 (100%) | 171 (97%) | 126 (97%) | 348 (97%) |

Patient demographics
Age

| | | | | | | | | |
|---|---|---|---|---|---|---|---|---|
| Median | 63 | [31, 84] | 58 | [35, 65] | 77 | [65, 92] | 64 | [31, 92] |
| [Min, Max] | | | | | | | | |

Seks

| | | | | | | | | |
|---|---|---|---|---|---|---|---|---|
| Female | 23 | (45%) | 67 | (39%) | 51 | (40%) | 141 | (41%) |
| Male | 28 | (55%) | 104 | (61%) | 75 | (60%) | 207 | (59%) |

CTC level (%)

| | | | | | | | | |
|---|---|---|---|---|---|---|---|---|
| Median | 31 | [2.0, 85] | 0.021 | [0, 26] | 0.012 | [0, 36] | 0.031 | [0, 85] |
| [Min, Max] | | | | | | | | |

BM plasmacytosis (%)

| | | | | | | | | |
|---|---|---|---|---|---|---|---|---|
| Median | 64 | [12, 100] | 31 | [0, 100] | 35 | [4, 97] | 35 | [0, 100] |
| [Min, Max] | | | | | | | | |

Anemia

| | | | | | | | | |
|---|---|---|---|---|---|---|---|---|
| Absent | 1 | (2%) | 31 | (18%) | 9 | (7%) | 41 | (12%) |
| Present | 50 | (98%) | 140 | (82%) | 117 | (93%) | 307 | (88%) |

Bone lesions

| | | | | | | | | |
|---|---|---|---|---|---|---|---|---|
| Absent | 21 | (41%) | 27 | (16%) | 28 | (23%) | 76 | (22%) |
| Present | 30 | (59%) | 144 | (84%) | 95 | (77%) | 269 | (78%) |

Hypercalcemia

| | | | | | | | | |
|---|---|---|---|---|---|---|---|---|
| Absent | 39 | (76%) | 161 | (95%) | 116 | (92%) | 316 | (91%) |
| Present | 12 | (24%) | 9 | (5%) | 10 | (8%) | 31 | (9%) |

Hypoalbuminemia

| | | | | | | | | |
|---|---|---|---|---|---|---|---|---|
| Absent | 31 | (61%) | 111 | (65%) | 66 | (52%) | 208 | (60%) |
| Present | 20 | (39%) | 60 | (35%) | 60 | (48%) | 140 | (40%) |

LDH (upper limit of normal)

| | | | | | | | | |
|---|---|---|---|---|---|---|---|---|
| <=ULN | 19 | (43%) | 140 | (83%) | 111 | (90%) | 270 | (80%) |
| >ULN | 25 | (57%) | 28 | (17%) | 13 | (10%) | 66 | (20%) |

Leukocytosis

| | | | | | | | | |
|---|---|---|---|---|---|---|---|---|
| Absent | 19 | (43%) | 162 | (95%) | 123 | (98%) | 304 | (89%) |
| Present | 25 | (57%) | 9 | (5%) | 3 | (2%) | 37 | (11%) |

Renal failure

| | | | | | | | | |
|---|---|---|---|---|---|---|---|---|
| Absent | 38 | (75%) | 171 | (100%) | 114 | (90%) | 323 | (93%) |
| Present | 13 | (25%) | 0 | (0%) | 12 | (10%) | 25 | (7%) |

Soft tissue plasmacytoma

| | | | | | | | | |
|---|---|---|---|---|---|---|---|---|
| Absent | 40 | (82%) | 171 | (100%) | 82 | (90%) | 293 | (94%) |
| Present | 9 | (18%) | 0 | (0%) | 9 | (10%) | 18 | (6%) |

Thrombocytopenia

| | | | | | | | | |
|---|---|---|---|---|---|---|---|---|
| Absent | 18 | (41%) | 156 | (91%) | 111 | (88%) | 285 | (84%) |
| Present | 26 | (59%) | 15 | (9%) | 15 | (12%) | 56 | (16%) |

Risk assessment
ISS stage

| | | | | | | | | |
|---|---|---|---|---|---|---|---|---|
| I | 5 | (11%) | 68 | (40%) | 26 | (21%) | 99 | (29%) |
| II | 10 | (22%) | 74 | (43%) | 59 | (47%) | 143 | (42%) |
| III | 31 | (67%) | 29 | (17%) | 40 | (32%) | 100 | (29%) |

R-ISS stage

| | | | | | | | | |
|---|---|---|---|---|---|---|---|---|
| I | 1 | (3%) | 39 | (25%) | 20 | (17%) | 60 | (19%) |
| II | 18 | (46%) | 102 | (66%) | 84 | (71%) | 204 | (66%) |
| III | 20 | (51%) | 13 | (8%) | 14 | (12%) | 47 | (15%) |

High-risk FISH

| | | | | | | | | |
|---|---|---|---|---|---|---|---|---|
| Absent | 17 | (53%) | 104 | (81%) | 90 | (84%) | 211 | (79%) |
| Present | 15 | (47%) | 25 | (19%) | 17 | (16%) | 57 | (21%) |

Cytogenetic aberrations
Hyperdiploidy

| | | | | | | | | |
|---|---|---|---|---|---|---|---|---|
| Absent | 19 | (90%) | 25 | (36%) | 19 | (28%) | 63 | (40%) |
| Present | 2 | (10%) | 45 | (64%) | 48 | (72%) | 95 | (60%) |

IgH translocation

| | | | | | | | | |
|---|---|---|---|---|---|---|---|---|
| Absent | 26 | (51%) | 118 | (69%) | 101 | (80%) | 245 | (70%) |
| Present | 25 | (49%) | 53 | (31%) | 25 | (20%) | 103 | (30%) |

Del1p32

| | | | | | | | | |
|---|---|---|---|---|---|---|---|---|
| Absent | 20 | (65%) | 79 | (92%) | 92 | (89%) | 191 | (87%) |
| Present | 11 | (35%) | 7 | (8%) | 11 | (11%) | 29 | (13%) |

Gain1q21

| | | | | | | | | |
|---|---|---|---|---|---|---|---|---|
| Absent | 19 | (70%) | 76 | (68%) | 73 | (70%) | 168 | (69%) |
| Present | 8 | (30%) | 35 | (32%) | 31 | (30%) | 74 | (31%) |

Del13q14

| | | | | | | | | |
|---|---|---|---|---|---|---|---|---|
| Absent | 12 | (38%) | 71 | (63%) | 31 | (46%) | 114 | (54%) |
| Present | 20 | (63%) | 41 | (37%) | 37 | (54%) | 98 | (46%) |

Del17p13

| | | | | | | | | |
|---|---|---|---|---|---|---|---|---|
| Absent | 20 | (61%) | 145 | (90%) | 101 | (91%) | 266 | (87%) |
| Present | 13 | (39%) | 17 | (10%) | 10 | (9%) | 40 | (13%) |

t(4; 14)

| | | | | | | | | |
|---|---|---|---|---|---|---|---|---|
| Absent | 37 | (95%) | 150 | (92%) | 111 | (97%) | 298 | (94%) |
| Present | 2 | (5%) | 13 | (8%) | 3 | (3%) | 18 | (6%) |

t(8; 14)

| | | | | | | | | |
|---|---|---|---|---|---|---|---|---|
| Absent | 8 | (73%) | 87 | (90%) | 31 | (97%) | 126 | (90%) |
| Present | 3 | (27%) | 10 | (10%) | 1 | (3%) | 14 | (10%) |

t(11; 14)

| | | | | | | | | |
|---|---|---|---|---|---|---|---|---|
| Absent | 20 | (50%) | 134 | (79%) | 89 | (83%) | 243 | (77%) |
| Present | 20 | (50%) | 36 | (21%) | 18 | (17%) | 74 | (23%) |

t(14; 16)

| | | | | | | | | |
|---|---|---|---|---|---|---|---|---|
| Absent | 35 | (92%) | 128 | (99%) | 108 | (96%) | 271 | (97%) |
| Present | 3 | (8%) | 1 | (1%) | 4 | (4%) | 8 | (3%) |

CTC immunophenotype
CD19

| | | | | | | | | |
|---|---|---|---|---|---|---|---|---|
| Negative | 12 | (100%) | 118 | (97%) | 83 | (95%) | 213 | (96%) |
| Positive | 0 | (0%) | 4 | (3%) | 4 | (5%) | 8 | (4%) |

CD27

| | | | | | | | | |
|---|---|---|---|---|---|---|---|---|
| Negative | 1 | (8%) | 30 | (25%) | 11 | (13%) | 42 | (19%) |
| Positive | 11 | (92%) | 92 | (75%) | 76 | (87%) | 179 | (81%) |

CD45

| | | | | | | | | |
|---|---|---|---|---|---|---|---|---|
| Negative | 6 | (50%) | 62 | (51%) | 31 | (36%) | 99 | (45%) |
| Positive | 6 | (50%) | 60 | (49%) | 56 | (64%) | 122 | (55%) |

CD56

| | | | | | | | | |
|---|---|---|---|---|---|---|---|---|
| Negative | 7 | (58%) | 32 | (26%) | 27 | (31%) | 66 | (30%) |
| Positive | 5 | (42%) | 90 | (74%) | 60 | (69%) | 155 | (70%) |

CD81

| | | | | | | | | |
|---|---|---|---|---|---|---|---|---|
| Negative | 11 | (92%) | 94 | (77%) | 75 | (86%) | 180 | (81%) |
| Positive | 1 | (8%) | 28 | (23%) | 12 | (14%) | 41 | (19%) |

CD117

| | | | | | | | | |
|---|---|---|---|---|---|---|---|---|
| Negative | 10 | (83%) | 85 | (70%) | 54 | (62%) | 149 | (67%) |
| Positive | 2 | (17%) | 37 | (30%) | 33 | (38%) | 72 | (33%) |

CD138

| | | | | | | | | |
|---|---|---|---|---|---|---|---|---|
| Negative | 12 | (100%) | 118 | (97%) | 87 | (100%) | 217 | (98%) |
| Positive | 0 | (0%) | 4 | (3%) | 0 | (0%) | 4 | (2%) |

CD38

| | | | | | | | | |
|---|---|---|---|---|---|---|---|---|
| Positive | 12 | (100%) | 122 | (100%) | 87 | (100%) | 221 | (100%) |
| Negative | 0 | (0%) | 0 | (0%) | 0 | (0%) | 0 | (0%) |

CyIgK

| | | | | | | | | |
|---|---|---|---|---|---|---|---|---|
| Negative | 6 | (50%) | 29 | (24%) | 27 | (31%) | 62 | (28%) |
| Positive | 6 | (50%) | 90 | (76%) | 60 | (69%) | 156 | (72%) |

CyIgL

| | | | | | | | | |
|---|---|---|---|---|---|---|---|---|
| Negative | 6 | (50%) | 87 | (73%) | 61 | (70%) | 154 | (71%) |
| Positive | 6 | (50%) | 32 | (27%) | 26 | (30%) | 64 | (29%) |

*Patients enrolled in the EMN12/HO129 trial who had a protocol start before Jul. 1, 2019 were included in this study.
**Only HOVON patients were eligible for this study.

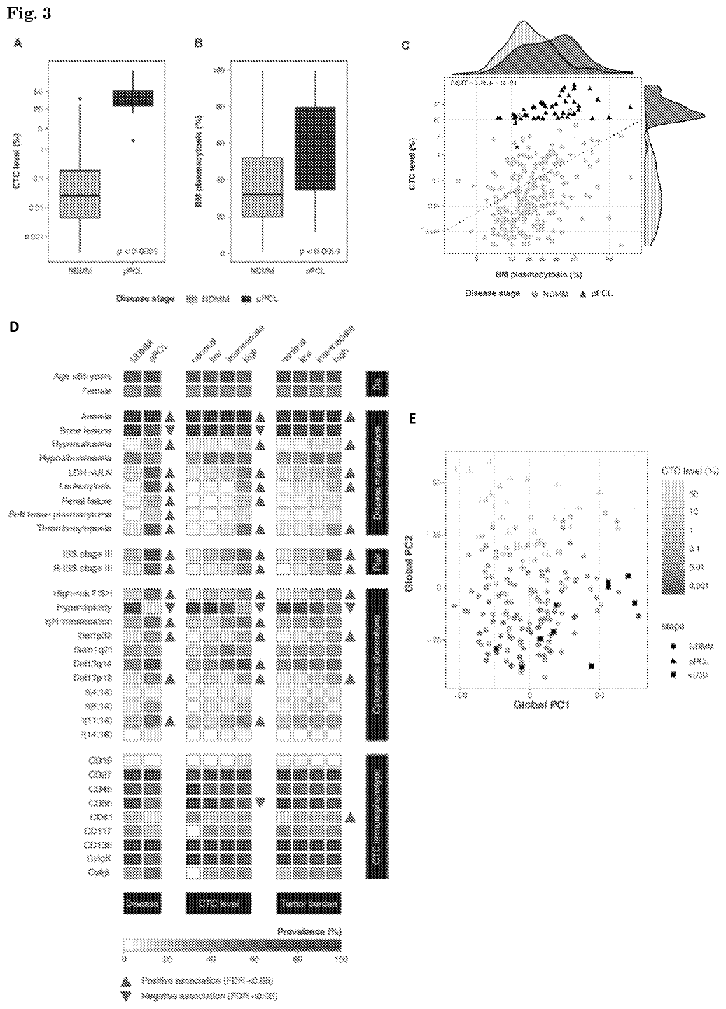

Baseline CTC levels (median, 31% versus 0.016%, p<0.0001) and tumor burden as reflected by BM plasmacytosis (median, 64% versus 32%, p<0.0001) were both higher in pPCL than in NDMM patients (FIG. 3A-3B). Tumor burden and CTC levels showed a positive, yet weak association (adjusted R2, 0.16, p<0.0001), with all pPCL samples having higher CTC levels than expected based on their tumor burden (FIG. 3C).

pPCL patients presented with significantly higher morbidity than NDMM patients, including more hypercalcemia (24% versus 6%), renal failure (25% versus 4%) and soft tissue plasmacytoma (18% versus 3%), yet a lower occurrence of bone lesions (59% versus 81%) (false discovery rate (FDR)<0.05) (FIG. 3D). Moreover, high-risk FISH status (47% versus 18%), the presence of an IgH translocation (49% versus 26%), dellp32 (35% versus 10%), del17p13 (39% versus 10%) and t (11;14) (50% versus 19%) were all more frequently detected in pPCL than in NDMM, whereas hyperdiploidy was less observed in pPCL (10% versus 68%) (FDR<0.05). Of note, 15/16 (94%) PCL-like features identified in this analysis were also significantly associated with CTC level (FDR<0.05), whereas 11/16 (69%) PCL-like features also correlated with tumor burden.

### Example 2: A Transcriptomic Profile Representing PCL-Like Disease

To enable a more comprehensive screening of tumor cell aberrations that associate with PCL-like disease, transcriptomic profiling was performed of BM tumor cells in a subgroup of 154 NDMM and 29 pPCL patients from cohort 1 (FIG. 1). In a global principal component analysis (PCA) using all 12,928 genes that were expressed in these 183 samples, pPCL samples clustered together. Yet, a subgroup of NDMM samples had a highly similar transcriptomic profile to pPCL samples and these generally had CTC levels that were above average for NDMM (FIG. 3E).

For the identification of essential genes defining this PCL-like transcriptome, cohort 1 was divided into a discovery (n=124) and validation set (n=59), including both NDMM and pPCL patients in each set (FIG. 1, Tables 1 and 5). To optimize the power to detect bona fide PCL-like genes, a linear model was applied, in which CTC level was used as a surrogate marker for PCL-likeness, rather than comparing pPCL with NDMM samples in a dichotomous model. After correction for tumor burden, 1700 genes were identified that had a significant association with CTC level in the discovery cohort (FDR<0.05). These genes were amongst others involved in cell adhesion (e.g. NCAM1, ITGA6, SDC1), tumor suppression (e.g. PTEN, TUSC2, TAGLN2), proliferation (e.g. MKI67, MCM2, CENPM), RNA splicing (e.g. SRSF10, SF3A2, PUF60), cell migration (e.g. ROCK1, DOCK11, DLC1) and DNA damage control (e.g. CHEK1, DCLRE1C, SLFN11).

TABLE 5
Used gene expression datasets

| | | | | | | | | |
|---|---|---|---|---|---|---|---|---|
| | | | | | N patients | | GEO | Other type |
| | Disease | Tumor | N samples | N samples | in survival | | accession | of accession |
| Dataset | stage | source | in dataset | in molecular analyses | analyses | Method | number | number |

Novel datasets

| | | | | | | | |
|---|---|---|---|---|---|---|---|
| Cassiopeia | NDMM | BM | 109 | 109 | 0 | Human Genome U133 | GSE164701 |
| | | | | | | Plus 2.0 Array | |
| | | | | | | (Affymetrix) | |
| HO143 | NDMM | BM | 45 | 45 | 0 | RNA Seq; mRNA | GSE164830 |
| | | | | | | HyperPrep Kit (KAPA) | |
| EMN12/HO129 | pPCL | BM | 29 | 29 | 0 | Human Genome U133 | GSE164703 |
| | | | | | | Plus 2.0 Array | |
| | | | | | | (Affymetrix) | |
| | pPCL | CTC | 28 | 28 | 0 | Human Genome U133 | |
| | | | | | | Plus 2.0 Array | |
| | | | | | | (Affymetrix) | |
| EMN02/HO95 | NDMM | BM | 240 | 240 | 240 | Human Genome U133 | GSE164706 |
| | | | | | | Plus 2.0 Array | |
| | | | | | | (Affymetrix) | |
| | NDMM | BM | 123 | 123 | 0 | RNA Seq; mRNA | GSE164847 |
| | | | | | | HyperPrep Kit (KAPA) | |

Publicly available datasets

| | | | | | | | | |
|---|---|---|---|---|---|---|---|---|
| HOVON-65/ | NDMM | BM | 328 | 327 | 327 | Human Genome U133 | GSE19784 | |
| GMMG-HD4 | | | | | | Plus 2.0 Array | | |
| | | | | | | (Affymetrix) | | |
| HOVON-87/ | NDMM | BM | 180 | 180 | 180 | Human Genome U133 | GSE87900 | |
| NMSG-18 | | | | | | Plus 2.0 Array | | |
| | | | | | | (Affymetrix) | | |
| MRC-IX | NDMM | BM | 247 | 234 | 234 | Human Genome U133 | GSE15695 | |
| | | | | | | Plus 2.0 Array | | |
| | | | | | | (Affymetrix) | | |
| Total | NDMM | BM | 345 | 345 | 345 | Human Genome U133 | GSE24080 | |
| Therapy 2 | | | | | | Plus 2.0 Array | | |
| | | | | | | (Affymetrix) | | |
| Total | NDMM | BM | 214 | 214 | 214 | | | |
| Therapy 3 | | | | | | | | |
| (A + B) | | | | | | | | |
| MMRF CoMMpass | NDMM | BM | 921 | 0 | 0 | RNA Seq; TruSeq RNA | | MMRF Researcher Gateway |
| | NDMM | CTC | | | | Library Prep Kit v2 | | (https://research.themmrf.org), |
| | PD | BM | | | | (Illumina) | | release IA15 |
| GSE5900 | Healthy plasma cells | BM | 22 | 22 | 0 | Human Genome U133 | GSE5900 | |
| | MGUS | BM | 44 | 44 | 0 | Plus 2.0 Array | | |
| | sMM | BM | 12 | 12 | 0 | (Affymetrix) | | |
| GSE159289 | MM cell lines | cell line | 4 | 4 | 0 | Human Genome U133 | GSE159289 | |
| | | | | | | Plus 2.0 Array | | |
| | | | | | | (Affymetrix) | | |

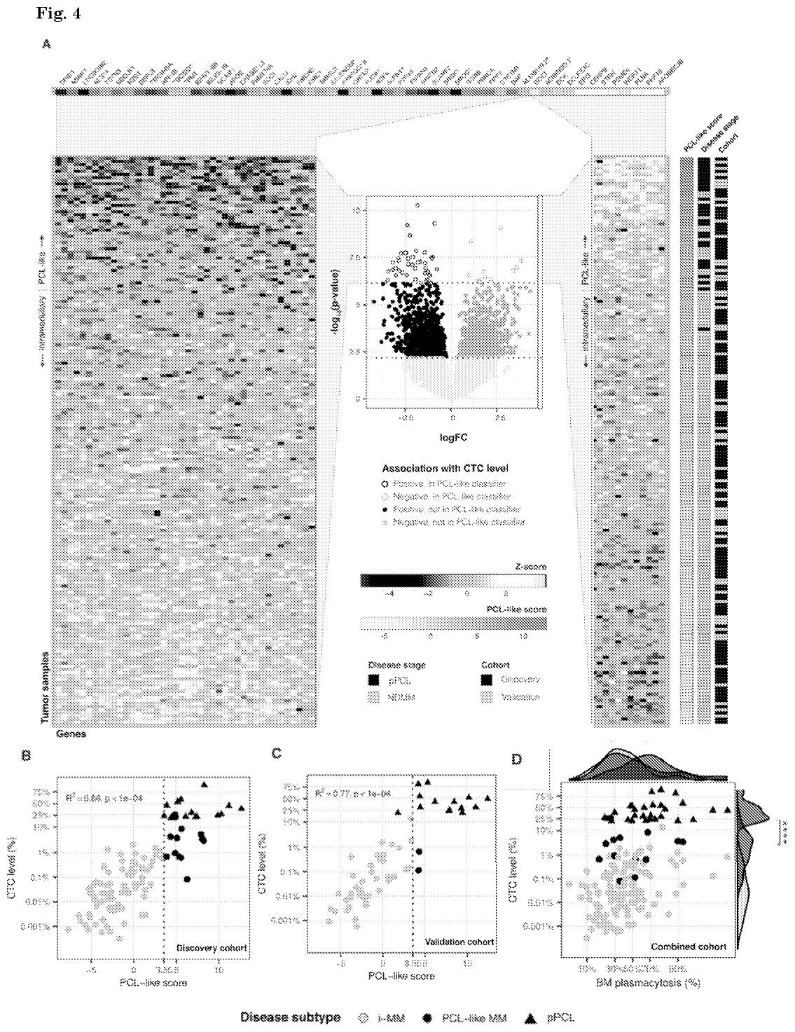

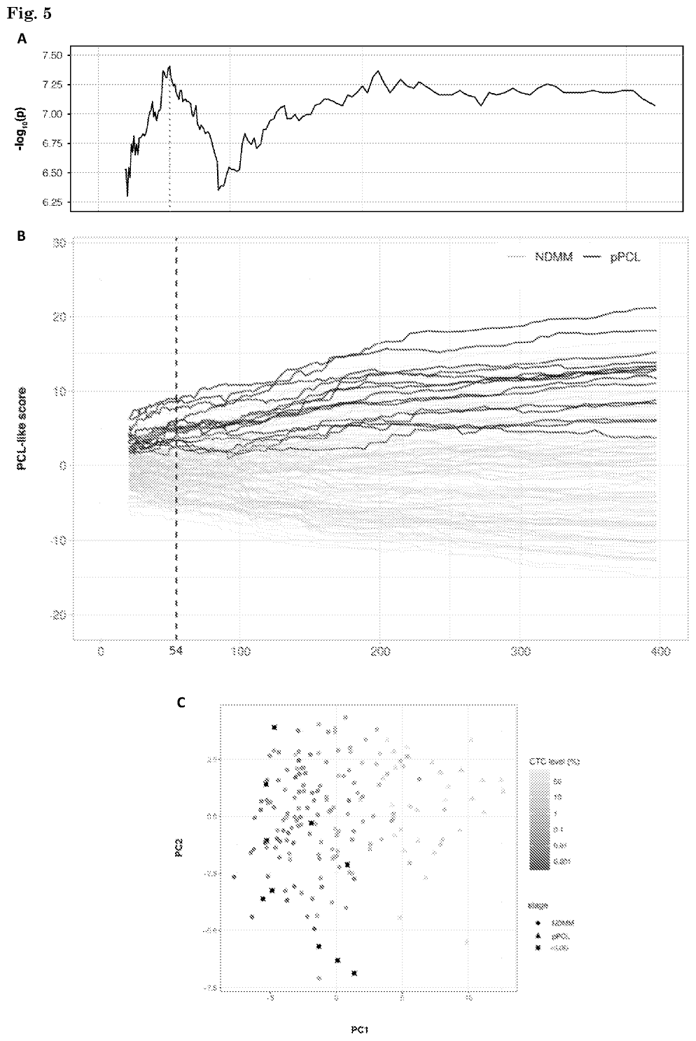

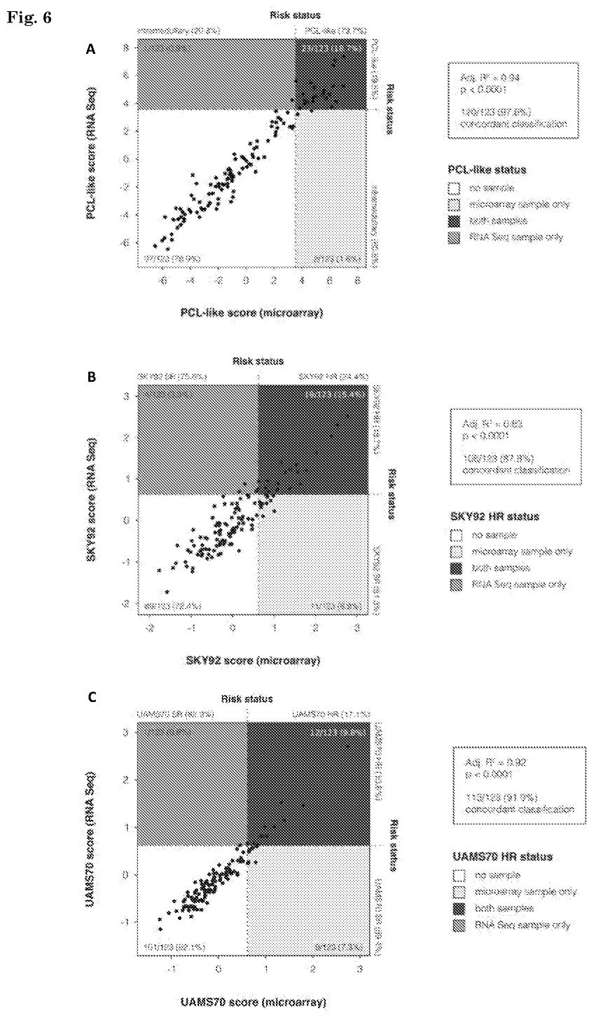

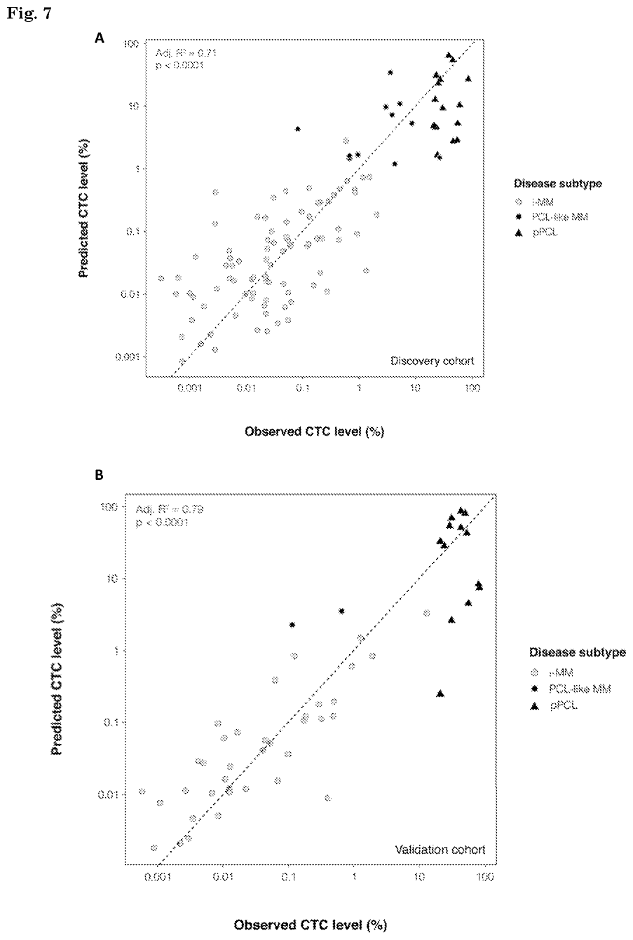

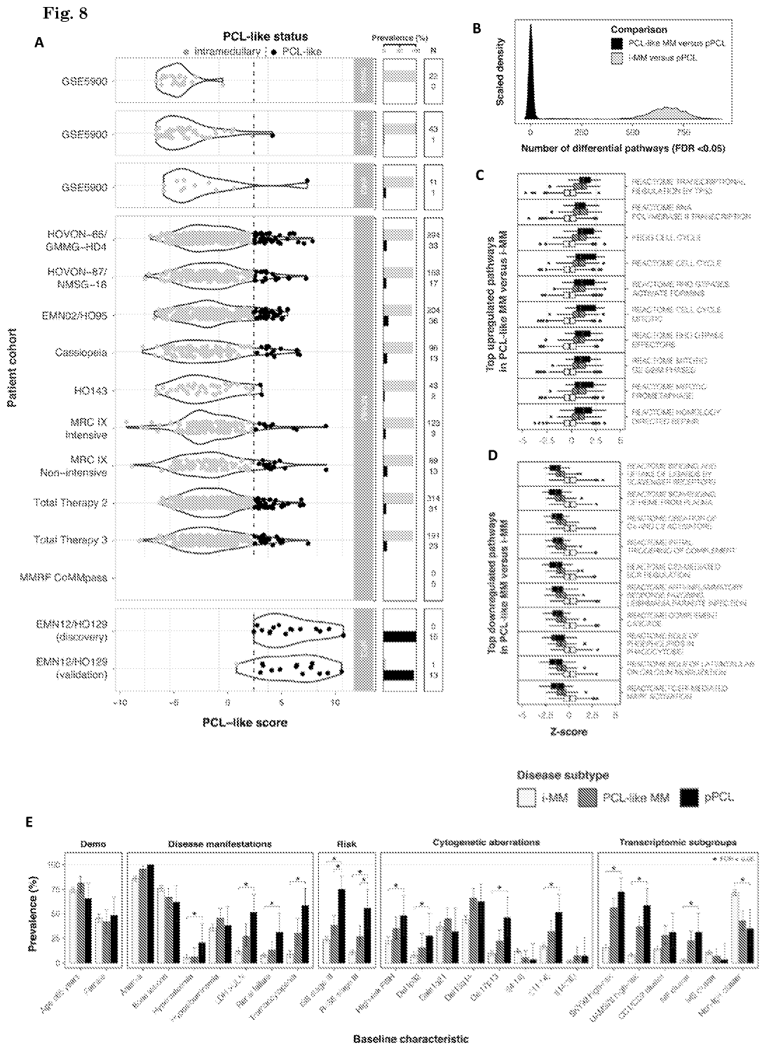

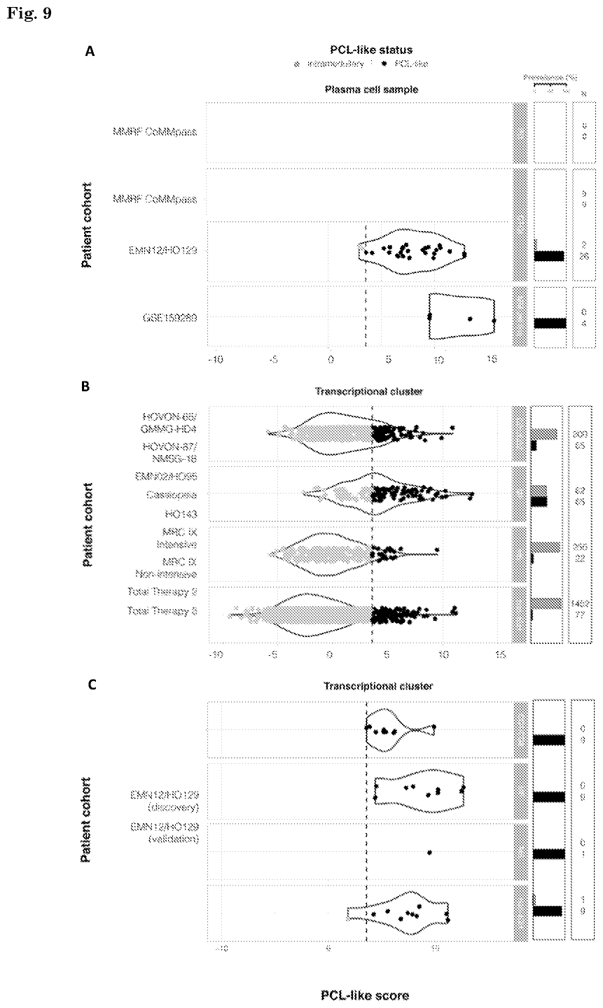

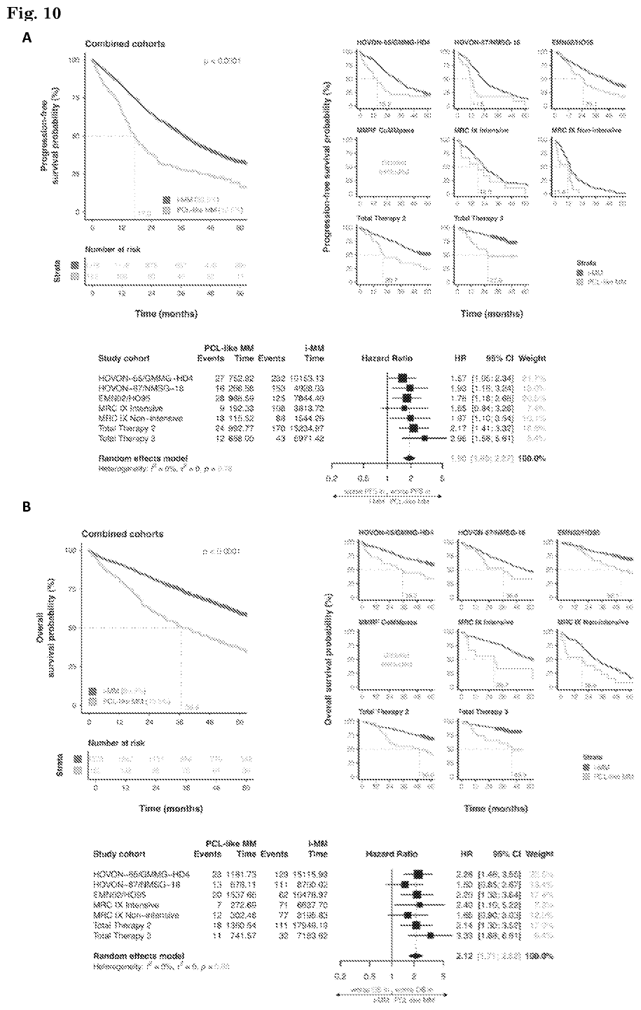

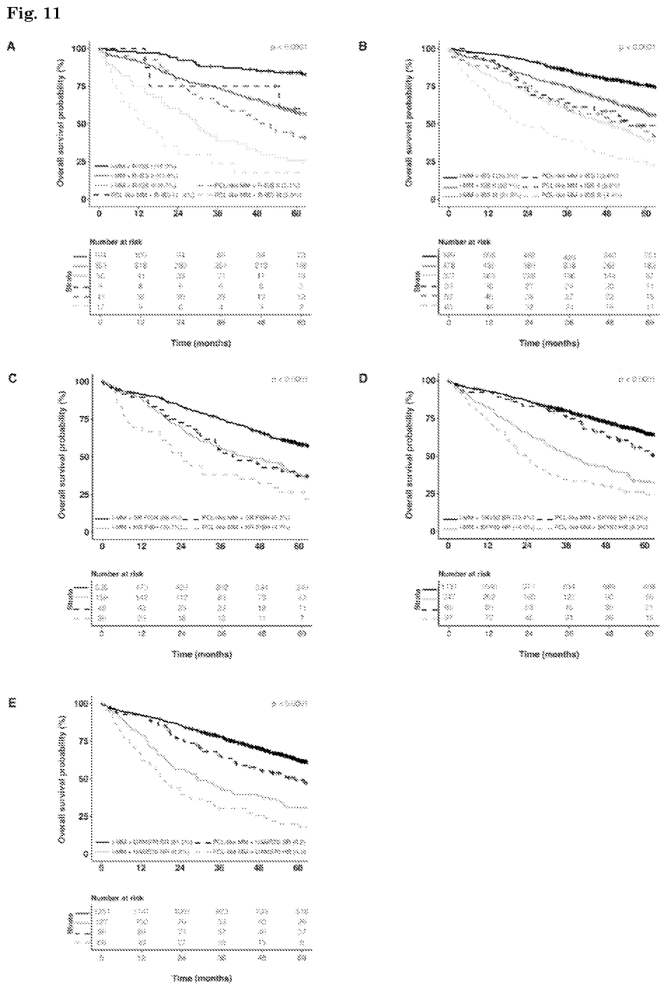

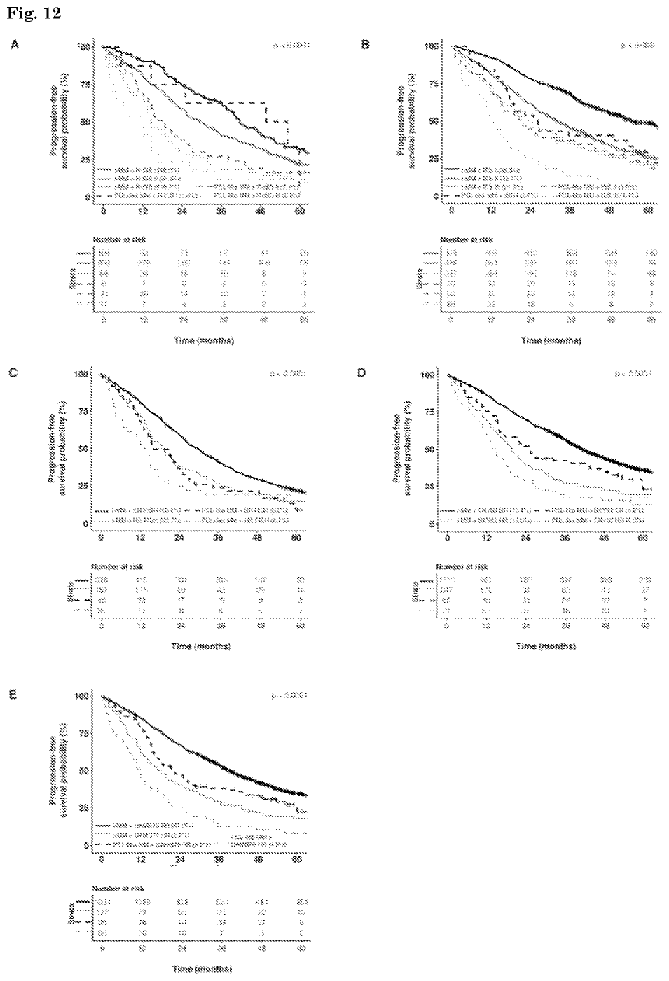

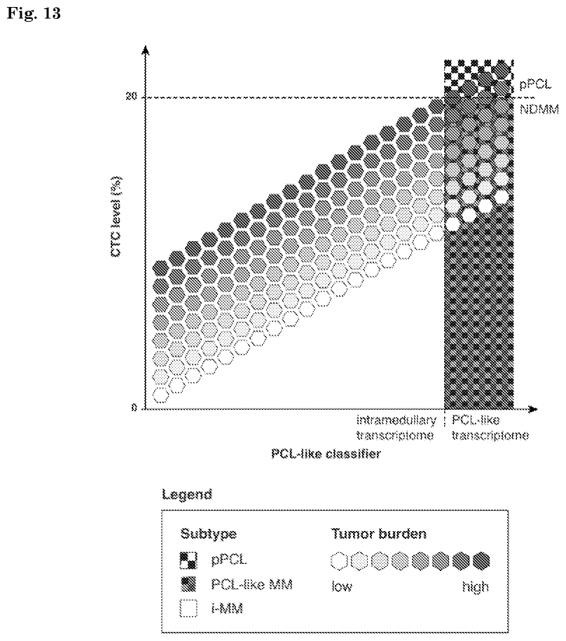

By using the composite information of a selection of 54/1700 genes, a score was constructed with which pPCL could be best distinguished from NDMM samples: the score (FIG. 4A, FIG. 5, Table 6). This score was independent of the platform that was used to generate it (microarray versus RNA Seq), as evidenced by a high inter-platform correlation of scores in 123 paired samples (adjusted R2, 0.94; p<0.0001) (FIG. 6, Tables 1 and 5). To validate that this score indeed represented PCL-like disease, a linear regression model was constructed to calculate predicted CTC levels for all patients based on both tumor burden and score. This showed that in the validation cohort, 60% of the variance in CTC levels could be predicted by the score and 6% by tumor burden, with observed CTC levels strongly correlating with predicted CTC levels (adjusted R2, 0.79; p <0.0001) (FIG. 7).

TABLE 6
PCL-like classifier genes

| Ensembl gene ID | Weight | Center | Gene symbol | Chromosome | Rand | Description | Source | Biological process |
|---|---|---|---|---|---|---|---|---|
| ENSG [symbol] | −0.35686 | 11.43547 | SOC1 | 2 | p [symbol] .1 | [symbol] 1 | Source HGNC Symbol Acc: [symbol] ] | Cell adhesion |
| ENSG [symbol] | −0.119 [symbol] | 8.9732 [symbol] | [symbol] -19 | 12 | q11.22 | [symbol] | Source HGNC Symbol Acc:5903] | immune response |
| ENSG [symbol] | −0.110 [symbol] | 12.8570 [symbol] | PP [symbol] 1B | 8 | p11.23 | [symbol] | Source HGNC Symbol Acc:35036] | Cell metabolism |
| ENSG [symbol] | −0. [symbol] 943 | 8. [symbol] | [symbol] | 10 | q [symbol] 2 | [symbol] | Source HGNC Symbol Acc:13851] | Tumor suppression |
| ENSG [symbol] | −0.113 [symbol] 4 | 11.84765 | [symbol] | 1 | p [symbol] | [symbol] subunit | Source HGNC Symbol Acc:28 [symbol] ] | Post [symbol] protein modification |
| ENSG [symbol] | −0.21353 | 8.32714 | [symbol] | 231 | q [symbol] 3.2 | [symbol] protein 19 | Source HGNC Symbol Acc:2456 [symbol] ] | (Post [symbol] regulation |
| ENSG [symbol] | −0.10 [symbol] | 8.23796 | [symbol] | 13 | q14.13 | [symbol] family member 1 | Source HGNC Symbol Acc:168 [symbol] ] | (Post [symbol] regulation |
| ENSG [symbol] | −0.172 [symbol] 4 | 18.31024 | [symbol] | 5 | q21.1 | [symbol] similarity [symbol] member A | Source HGNC Symbol Acc:2 [symbol] 943] | Cellular (matrix) structure |
| ENSG [symbol] | −0. [symbol] | 12. [symbol] | [symbol] | 15 | q24.3 | [symbol] 3 | Source HGNC Symbol Acc:17752] | Cell migration |
| ENSG [symbol] | −0. [symbol] 4 | 11. [symbol] | [symbol] | 7 | q [symbol] .1 | [symbol] | Source HGNC Symbol Acc:1450] | [symbol] protein modification |
| ENSG [symbol] | −0. [symbol] 82 | 8.0236 [symbol] | [symbol] | 15 | q22.2 | [symbol] | Source HGNC Symbol Acc:120 [symbol] ] | Cell death |
| ENSG [symbol] | −0. [symbol] | [symbol] | [symbol] | 1 | p21.2 | [symbol] molecule 1 | Source HGNC Symbol Acc:12683] | Cell adhesion |
| ENSG [symbol] | −0. [symbol] | 10. [symbol] | [symbol] | 15 | q26.1 | [symbol] mitochondrial | Source HGNC Symbol Acc:5383] | Cell metabolism |
| ENSG [symbol] | −0. [symbol] | 2. [symbol] | [symbol] | 12 | q13.4 | [symbol] | Source HGNC Symbol Acc:8543] | Immune response |
| ENSG [symbol] | −0. [symbol] | 12. [symbol] | [symbol] | 8 | p22 | [symbol] 1 | Source HGNC Symbol Acc: [symbol] ] | Cell migration |
| ENSG [symbol] | −0. [symbol] 20 | 2. [symbol] | [symbol] | 14 | q32.33 | [symbol] | Source HGNC Symbol Acc: [symbol] ] | Immune response |
| ENSG [symbol] | −0. [symbol] 94 | 13. [symbol] | [symbol] | 1 | p [symbol] 11 | [symbol] | Source HGNC Symbol Acc:4006] | Tumor suppression |
| ENSG [symbol] | −0. [symbol] | [symbol] .75104 | [symbol] | 1 | p [symbol] | [symbol] binding protein | Source HGNC Symbol Acc:11424] | Cell signaling |
| ENSG [symbol] | −0. [symbol] | 11. [symbol] | [symbol] | 5 | q [symbol] | [symbol] 1 | Source HGNC Symbol Acc: [symbol] 283] | Immune response |
| ENSG [symbol] | −0. [symbol] | 8.93157 | [symbol] | 15 | p [symbol] 2.2 | [symbol] subunit | Source HGNC Symbol Acc:24 [symbol] 0] | Cell signaling |
| ENSG [symbol] | −0. [symbol] | 13. [symbol] | [symbol] | 1 | q23.3 | [symbol] family member 7 | Source HGNC Symbol Acc:21] | Immune response |
| ENSG [symbol] | −0.114 [symbol] | 12.53408 | [symbol] | 5 | q31.3 | [symbol] family member 5 | Source HGNC Symbol Acc:24872] | Protein biogenesis and transport |
| ENSG [symbol] | −0. [symbol] | 8. [symbol] | [symbol] | 18 | q13.32 | [symbol] E | Source HGNC Symbol Acc:813] | Cell metabolism |
| ENSG [symbol] | −0.02135 | 10.8 [symbol] | [symbol] | 12 | q13.12 | [symbol] | Source HGNC Symbol Acc:18850] | Cell proliferation |
| ENSG [symbol] | −0. [symbol] | 2.58174 | [symbol] | 17 | q [symbol] | [symbol] alpha | Source HGNC Symbol Acc: [symbol] 393] | Cell proliferation |
| ENSG [symbol] | −0. [symbol] | 8. [symbol] | [symbol] | 2 | p16 [symbol] | [symbol] subunit [symbol] | Source HGNC Symbol Acc:206 [symbol] ] | DNA damage response |
| ENSG [symbol] | −0. [symbol] | 8. [symbol] | [symbol] | 17 | q21 | [symbol] family member 11 | Source HGNC Symbol Acc:26633] | Cell death |
| ENSG [symbol] | −0. [symbol] | 13.0 [symbol] | [symbol] | 15 | q15.1 | [symbol] 3 | Source HGNC Symbol Acc: [symbol] 550] | Cell death |
| ENSG [symbol] | −0. [symbol] | 12. [symbol] | [symbol] | 12 | q15.5 | [symbol] domain containing [symbol] | Source HGNC Symbol Acc:28474] | Immune response |
| ENSG [symbol] | −0. [symbol] | 13. [symbol] | [symbol] | 3 | q [symbol] 2 | [symbol] proein [symbol] | Source HGNC Symbol Acc: [symbol] ] | Cell proliferation |
| ENSG [symbol] | −0. [symbol] | 8. [symbol] | [symbol] | 15 | q [symbol] 6.3 | [symbol] 2 | Source HGNC Symbol Acc:24 [symbol] ] | Protein biogenesis and transport |
| ENSG [symbol] | −0. [symbol] 23 | 7. [symbol] | [symbol] | 10 | p13 | [symbol] 1C | Source HGNC Symbol Acc:17643] | DNA damage response |
| ENSG [symbol] | −0. [symbol] 11 | 10. [symbol] | [symbol] | 10 | q24.1 | [symbol] family member 3 | Source HGNC Symbol Acc:24 [symbol] ] | Cell death |
| ENSG [symbol] | −0. [symbol] | 11.73273 | [symbol] | 5 | p [symbol] | [symbol] | Source HGNC Symbol Acc: [symbol] ] | Cell death |
| ENSG [symbol] | −0. [symbol] 1 | 8.6242 [symbol] | [symbol] | 4 | q13.3 | [symbol] kinase | Source HGNC Symbol Acc:22 [symbol] ] | Cell metabolism |
| ENSG [symbol] | −0. [symbol] 1 | [symbol] | [symbol] | 14 | q24.2 | SPARC related [symbol] calcium binding [symbol] | Source HGNC Symbol Acc: [symbol] ] | Cell metabolism |
| ENSG [symbol] | −0. [symbol] 8 | 13. [symbol] | [symbol] | 15 | q14 | [symbol] protein complex subunit 7 | Source HGNC Symbol Acc: [symbol] ] | Protein biogenesis and transport |
| ENSG [symbol] | −0. [symbol] 9 | 10. [symbol] | [symbol] | 1 | p42.1 | [symbol] | Source HGNC Symbol Acc: [symbol] ] | (Post [symbol] regulation |
| ENSG [symbol] | −0. [symbol] | 12.0 [symbol] | [symbol] | 19 | q13. [symbol] | [symbol] receptor 1 | Source HGNC Symbol Acc: [symbol] ] | Post [symbol] protein modification |
| ENSG [symbol] | −0. [symbol] 1 | 12. [symbol] | [symbol] | 22 | q12.2 | [symbol] M | Source HGNC Symbol Acc: [symbol] ] | Cell migration |
| ENSG [symbol] | −0. [symbol] 41 | 10.1 [symbol] | [symbol] | 32 | q13.1 | [symbol] 3B | Source HGNC Symbol Acc:17 [symbol] ] | (Post [symbol] regulation |
| ENSG [symbol] | −0. [symbol] 88 | 11. [symbol] | [symbol] | 1 | p32.1 | [symbol] protein | Source HGNC Symbol Acc:2539] | Cellular(matrix) structure |
| ENSG [symbol] | −0. [symbol] 17 | 7.4 [symbol] | [symbol] | 39 | q13 [symbol] | [symbol] kinase 1 | Source HGNC Symbol Acc: [symbol] ] | DNA damage response |
| ENSG [symbol] | −0. [symbol] 24 | 6. [symbol] | [symbol] | [symbol] | q [symbol] | | | (Post [symbol] regulation |
| ENSG [symbol] | −0. [symbol] 34 | 12. [symbol] | [symbol] | 5 | q [symbol] .2 | marginal [symbol] cell-specific protein | Source HGNC Symbol Acc:10325] | Immune response |
| ENSG [symbol] | −0. [symbol] 72 | 7. [symbol] | [symbol] | 1 | p14.1 | [symbol] family member 3 | Source HGNC Symbol Acc:17276] | (Post [symbol] regulation |
| ENSG [symbol] | −0. [symbol] 85 | 12. [symbol] | [symbol] | 22 | q11.23 | [symbol] 3 | Source HGNC Symbol Acc:14238] | Tumor suppression |
| ENSG [symbol] | −0. [symbol] 13 | 7. [symbol] | [symbol] | 22 | p13.3 | [symbol] protein M | Source HGNC Symbol Acc:18352] | Cell proliferation |
| ENSG [symbol] | −0. [symbol] 82 | 11. [symbol] | [symbol] | 16 | p12.3 | [symbol] 1 | Source HGNC Symbol Acc:19644] | Cell metabolism |
| ENSG [symbol] | −0. [symbol] | 8. [symbol] | [symbol] | 8 | q28 | [symbol] A alpha | Source HGNC Symbol Acc:1354] | Cellular (matrix) structure |
| ENSG [symbol] | −0. [symbol] 02 | [symbol] | [symbol] | 8 | p [symbol] | | | (Post [symbol] regulation |
| ENSG [symbol] | −0. [symbol] 86 | 10.0 [symbol] | [symbol] | 28 | q12.3 | [symbol] | Source HGNC Symbol Acc: [symbol] ] | Cell signaling |
| ENSG [symbol] | −0. [symbol] 93 | 7. [symbol] | [symbol] | 3 | p14.3 | [symbol] 3 | Source HGNC Symbol Acc:39 [symbol] ] | Cell death |
| ENSG [symbol] | −0. [symbol] 14 | 8. [symbol] | [symbol] | 10 | p13 | [symbol] alpha [symbol] | Source HGNC Symbol Acc: [symbol] 144] | Cell adhesion |

[symbol]

indicates data missing or illegible when filed

### Example 3: Identification of PCL-Like MM Tumors

Since the score is a reflection of PCL-like disease, it was hypothesized that this information could be leveraged to identify NDMM tumors with a similar transcriptome to pPCL tumors. To this end, a threshold for the PCL-like classifier was set by selecting the minimal score to include all pPCL tumors in the discovery cohort (FIG. 4B). With this threshold, 13/14 (93%) pPCL tumors in the validation cohort were correctly classified as “PCL-like” (FIG. 4C). Of note, a subgroup of NDMM tumors was also classified as “PCL-like” based on this threshold, despite presenting with CTC levels as low as 0.083%: PCL-like MM (FIG. 4B-C). PCL-like MM had both lower CTC levels (median, 3.0% versus 35%; p<0.0001) and a lower tumor burden (median, 36% versus 71%; p=0.045) than pPCL (FIG. 4D). NDMM patients who had a PCL-like score below 3.55 were referred to as intramedullary MM (i-MM).

To explore the prevalence of a PCL-like transcriptome in all stages of plasma cell malignancies, PCL-like status was determined in 1650 additional plasma cell samples (cohort 2) (FIG. 1, Table 5). In all nine NDMM cohorts a PCL-like transcriptome was consistently identified, with a prevalence ranging from 2/45 (4%) (HO143 cohort) to 36/240 (15%) (EMN02/HO95 cohort). PCL-like transcriptomics were not detected in healthy plasma cell samples, in 1/44 (2%) MGUS samples and in 1/12 (8%) SMM samples (FIG. 8A). 4/4 (100%) MM cell lines were classified as PCL-like, 26/28 (93%) CTC samples (FIG. 9A). Dividing NDMM and pPCL samples into four subgroups based on previously reported transcriptomic clusters, showed an enrichment of PCL-like transcriptomic status in the MF (55/99, 56%) and CD1/CD2 (57/275, 21%) clusters (FIG. 9B-9C).

### Example 4: Molecular and Clinical Determinants of PCL-Like MM

To further characterize PCL-like MM, additional data were collected for 885 NDMM and pPCL patients from cohort 2. First, single sample gene set enrichment analysis (ssGSEA) scores were generated for each tumor sample, including 1788 canonical pathways. A comparison of these ssGSEA scores between subgroups showed that pPCL and i-MM were highly distinct at the transcriptomic level, whereas PCL-like MM and pPCL were very similar (FIG. 7B). A total of 1160 pathways were differentially expressed between PCL-like MM and i-MM, which were amongst others involved in TP53 signaling, Rho GTPase activity, mitosis and binding and uptake of ligands (FIG. 8C-8D).

Also at the clinical and cytogenetic level, PCL-like MM was more similar to pPCL than i-MM. PCL-like MM only had a lower prevalence of R-ISS stage III (26% versus 56%) and ISS stage III (38% versus 75%) than pPCL, whereas i-MM differed from pPCL with respect to the presence of 14/25 investigated baseline characteristics, including del1p32 (8% versus 27%), del17p13 (10% versus 46%) and t (11;14) (17% versus 52%) (FDR<0.05) (FIG. 8E).

Of 28 pPCL patients, matched tumor samples from BM and PB were available. CTCs had a higher score than matched BM tumor samples (median, 7.42 versus 7.02, p=0.0045).

### Example 5: PCL-Like Transcriptomic Status as Independent Prognostic Marker in NDMM

To investigate whether PCL-like transcriptomic status could be used as a novel molecular high-risk factor in NDMM, its association with PFS and OS was evaluated in 1540 NDMM patients from seven different phase 2 and 3 trial cohorts (FIG. 1; Tables 3 and 5). This combined cohort had a median follow-up time of 63.4 months, with 162/1540 (11%) patients being classified as PCL-like. Overall, PCL-like transcriptomic status conferred both a significantly worse PFS (HR, 1.9; 95% CI, 1.60-2.27) and OS (HR, 2.12; 95% CI: 1.71-2.62) in univariate meta-analyses (FIGS. 10A and 10B). This negative prognostic impact was largely irrespective of the received treatment, with the highest impact on PFS and OS observed in the Total Therapy 3 trial cohort, in which PCL-like status had a HR of 2.96 (95% CI: 1.56-5.61) and 3.33 (95% CI: 1.68-6.61), respectively.

Multivariate regression analysis was performed to test if PCL-like transcriptomic status retained its prognostic value in the context of conventional high-risk markers in NDMM, independent of age and received treatment. This showed that PCL-like transcriptomic status significantly associated with both PFS and OS in the context of R-ISS stage, ISS stage, high-risk FISH, SKY92 high-risk status and UAMS70 high-risk status (Table 7, FIG. 11, FIG. 12).

TABLE 7
Multivariate analyses of prognostic factors for
progression-free and overall survival in NDMM

| | Progression-free survival | | Overall survival | |
|---|---|---|---|---|
| | Hazard Ratio | | Hazard Ratio | |
| Prognostic factor | (95% CI) | P-value | (95% CI) | P-value |
| PCL-like classifier: | 1.50 (1.12-2.01) | 0.007 | 1.72 (1.23-2.41) | 0.001 |
| PCL-like MM versus i-MM | | | | |
| Revised International | | | | |
| Staging System (R-ISS) | | | | |
| R-ISS II versus R-ISS I | 1.47 (1.14-1.69) | 0.003 | 2.32 (1.56-3.44) | <0.0001 |
| R-ISS III versus R-ISS I | 2.52 (1.80-3.53) | <0.0001 | 5.75 (3.64-9.08) | <0.0001 |
| Age: ≤65 years versus >65 | 0.65 (0.21-1.98) | 0.44 | 0.21 (0.05-0.85) | 0.03 |
| years | | | | |
| PCL-like classifier: | 1.85 (1.53-2.25) | <0.0001 | 1.85 (1.49-2.30) | <0.0001 |
| PCL-like MM versus i-MM | | | | |
| International | | | | |
| Staging System (ISS) | | | | |
| ISS II versus ISS I | 1.47 (1.26-1.72) | <0.0001 | 1.64 (1.34-2.01) | <0.0001 |
| ISS III versus ISS I | 1.81 (1.53-2.13) | <0.0001 | 2.52 (2.05-3.10) | <0.0001 |
| Age: ≤65 years versus >65 | 0.97 (0.77-1.23) | 0.83 | 0.86 (0.65-1.14) | 0.28 |
| years | | | | |
| PCL-like classifier: | 1.56 (1.20-2.02) | 0.0007 | 1.78 (1.33-2.36) | <0.0001 |
| PCL-like MM versus i-MM | | | | |
| FISH: high-risk versus | 1.52 (1.26-1.82) | <0.0001 | 1.86 (1.51-2.30) | <0.0001 |
| standard-risk | | | | |
| Age: ≤65 years versus >65 | 0.93 (0.65-1.35) | 0.71 | 0.70 (0.45-1.08) | 0.10 |
| years | | | | |
| PCL-like classifier: | 1.48 (1.22-1.80) | <0.0001 | 1.45 (1.16-1.81) | 0.0009 |
| PCL-like MM versus i-MM | | | | |
| SKY92 classifier: high- | 2.09 (1.80-2.42) | <0.0001 | 2.83 (2.39-3.36) | <0.0001 |
| risk versus standard-risk | | | | |
| Age: ≤65 years versus >65 | 0.68 (0.68-1.06) | 0.20 | 0.75 (0.57-0.98) | 0.04 |
| years | | | | |
| PCL-like classifier: | 1.66 (1.37-2.02) | <0.0001 | 1.60 (1.28-1.99) | <0.0001 |
| PCL-like MM versus i-MM | | | | |
| UAMS70 classifier: high-risk | 2.16 (1.81-2.57) | <0.0001 | 2.91 (2.39-3.54) | <0.0001 |
| versus standard-risk | | | | |
| Age: ≤65 years versus >65 | 0.69 (0.71-1.12) | 0.30 | 0.78 (0.59-1.02) | 0.07 |
| years | | | | |

PCL-like MM patients with R-ISS stage III (17/579, 3%) had a median OS of 13.7 months (95% CI, 6.8-41.1) versus not reached (95% CI, 87.8-NA) for i-MM patients with R-ISS stage I (104/579, 18%). Moreover, PCL-like MM patients with SKY92 high-risk disease (97/1540, 6%) had a median OS of 23.9 months (95% CI: 18.8-30.4) versus 87.8 months (95% CI: 81.2-NA) for i-MM patients with SKY92 standard-risk disease (1131/1540, 73%).

1. A marker set for determining a PCL-like transcriptomic status in a sample which is indicative for a disease
wherein the marker set comprises coding or non-coding genes associated to biological pathways and/or chromosomal location.

2. The marker set according to claim 1, wherein the disease is a rare disease and/or wherein the marker set indicates a high grading of the disease.

3. The marker set according to claim 1, wherein the marker set is selected from the group consisting of cell adhesion marker, immune response marker, cell metabolism marker, tumor suppression marker, post-translational protein modification marker, (post-) transcriptional regulation marker, cellular (matrix) structure marker, cell migration marker, cell death marker, cell signaling marker, protein biogenesis and transport marker, cell proliferation marker, and DNA damage response marker, or a combination thereof.

4. The marker set according to claim 1, wherein the marker set is selected from the group of markers consisting of SDC1, IGLV3-19, PPAPDC1B, WDR11, ALG14, PHF19, TSC22D1, FAM174A, TSPAN3, CALU, TPM1, VCAM1, IDH2, P2RY6, ASAH1, IGHV1-69, FUCA1, STRN, CYSTM1, APH1B, SLAMF7, YIPF5, APOE, SPATS2, PRKCA, PSME4, SLFN11, RMDN3, CHID1, TMEM45A, TARSL2, DCLRE1C, TCTN3, DAP, DCK, SMOC1, EMC7, LINC00582, KDELR1, APOBEC3B, CRTAP, BRSK1, MZB1, ERI3, DERL3, CENPM, GDE1, FLNA, NCF4, DNASEIL3, ITGA8, SELENOM, AL159169.2, and AC092620.1, or a combination thereof.

5. The marker set according to claim 4, wherein the marker set comprises a combination of two or more markers from the group of markers, optionally wherein the marker set comprises all the markers from the group of markers.

6. The marker set according to claim 1, wherein the sample is selected from plasma cell, blood, (pre-) malignant plasma cell, bone marrow, urine, serum, cells and tissue such as tumor tissue or tumor cells, or a combination thereof.

7. A method for determining a PCL-like transcriptomic status in a sample which is indicative for a disease comprising the steps of
a) isolating RNA from the sample
b) determining the expression profile of the marker set according to claim 1 in the isolated RNA,
c) calculating a score, wherein the score is based on the first principal component of the expression profile of the marker set in a classifier's discovery data,
d) comparing the score calculated in step c) to a reference score.

8. The method according to claim 7, wherein the score of step c) is the lowest score that at least 90 to 100% of the samples in a reference have a higher score.

9. The method according to claim 7, wherein a score of step c) in the range of at least 1 to 7 is indicative for a disease corresponding to the disease of the reference of step d).

10. The method according to claim 7, further comprising the steps of
e) determining the CTC level in the sample, and
f) optionally determining the tumor burden
g) referencing the expression profile of step b) to the CTC level or to the CTC level referenced to the tumor burden.

11. The method according to claim 7, wherein the PCL-like transcriptomic status indicates a high grading of a disease correlating to at least one prognostic risk model.

12. The method according to claim 7, further comprising selecting an active agent, such as a chemotherapeutic, for treatment of a disease based on the PCL-like transcriptomic status in a sample.

13. The marker set according to claim 1, wherein the disease is selected from the group consisting of newly diagnosed multiple myeloma (NDMM), primary plasma cell leukemia (pPCL), secondary plasma cell leukemia (pPCL), progressive disease (PD), smoldering multiple myeloma (SMM), monoclonal gammopathy of undetermined significance (MGUS), plasmacytomas, Waldenström's macroglobulinemia, POEMS syndrome, breast cancer, lung cancer, malignant melanoma, lymphoma, skin cancer, bone cancer, prostate cancer, liver cancer, brain cancer, cancer of the larynx, gall bladder, pancreas, testicular, rectum, parathyroid, thyroid, adrenal, neural tissue, head and neck, colon, stomach, bronchi, kidneys, basal cell carcinoma, squamous cell carcinoma, metastatic skin carcinoma, osteo sarcoma, Ewing's sarcoma, reticulum cell sarcoma, liposarcoma, myeloma, giant cell tumor, small-cell lung tumor, islet cell tumor, primary brain tumor, meningioma, acute and chronic lymphocytic and granulocytic tumors, acute and chronic myeloid leukemia, hairy-cell tumor, adenoma, hyperplasia, medullary carcinoma, intestinal ganglioneuromas, Wilms tumor, seminoma, ovarian tumor, leiomyomatous tumor, cervical dysplasia, retinoblastoma, soft tissue sarcoma, malignant carcinoid, actinic keratosis, melanoma, pancreatic cancer, colon cancer, rhabdomyosarcoma, Kaposi's sarcoma, osteogenic sarcoma, malignant hypercalcemia, renal cell tumor, polycythermia vera, myeloproliferative disease, essential thrombocytosis, lymphoma, mastocytosis, myelodysplastic syndrome, clonal hematopoiesis of indeterminate potential, monoclonal B-cell lymphocytosis, chronic myelomonocytic leukemia, myelofibrosis, adenocarcinoma, anaplastic astrocytoma, glioblastoma multiforma, epidermoid carcinoma, and a disease characterized by a circulating tumor cell, or a combination thereof.

14. The method according to claim 7, further comprising classifying the sample as having a high or standard SKY92 risk status, comprising determining in the sample the expression profile of each marker listed in Table 7.

15. A method for determining a treatment or prognosis for an individual afflicted with multiple myeloma, comprising:
determining a PCL-like transcriptomic status in a sample from said individual according to the method of claim 7,
determining the SKY92 risk status in a sample from said individual, comprising determining in the sample the expression profile of each marker listed in Table 7, and classifying the individual as having a high or standard SKY92 risk status.

16. A method for treating an individual afflicted with multiple myeloma, comprising:
determining a PCL-like transcriptomic status in a sample from said individual according to the method of claim 7, and
treating the individual by providing a cancer treatment to said individual.

17. A method for treating an individual afflicted with multiple myeloma, comprising:
a) determining a PCL-like transcriptomic status in a sample from said individual according to the method of claim 7,
b) determining the SKY92 risk status in a sample from said individual, comprising determining in the sample the expression profile of each marker listed in Table 7,
c) classifying said individual as having a PCL-like transcriptomic status and/or having a SKY92 high risk status, and
d) treating the individual of step c) by providing a cancer treatment to said individual.

18. The method of claim 16, wherein an individual classified as having a PCL-like transcriptomic status and optionally a SKY92 high risk status is intensively monitored, and treated with quadruplet induction therapy including anti-CD38, high dose autologous stem cell transplantation therapy or a combination thereof.

19. The method of claim 18, wherein a bispecific antibody, a CAR T cell or a combination thereof is administered.

20. Kit for determining a PCL-like transcriptomic status which is indicative for a disease comprising,
probes, primers, or a combination thereof for determining an expression profile of a marker set in a sample according to claim 1,
optionally means for determining the CTC level in a sample and
optionally means for determining the tumor burden in a sample.
